(19) United States
(12) Patent Application Publication
(10) Pub. No.: US 2025/0382608 A1
(43) Pub. Date: Dec. 18, 2025

(54) LINEAR NUCLEIC ACID EXPRESSION CONSTRUCTS

(71) Applicant: NUCLERA LTD., Cambridge, Cambridgeshire (GB)

(72) Inventors: James Allum, Cambridge, Cambridgeshire (GB); Adokiye Berepiki, Cambridge, Cambridgeshire (GB); Michael Chun Hao Chen, Cambridge, Cambridgeshire (GB); Namita Khanna, Cambridge, Cambridgeshire (GB); Tobias William Barr Ost, Cambridge, Cambridgeshire (GB)

(21) Appl. No.: 18/867,488

(22) PCT Filed: May 30, 2023
(86) PCT No.: PCT/GB2023/051412

§ 371 Date: Nov. 20, 2024

(30) Foreign Application Priority Data
May 27, 2022 (GB) 2207818.2

(51) Int. Cl. C12N 15/10; C07K 14/775; C12P 21/02

(52) U.S. Cl. CPC C12N 15/1093; C07K 14/775; C12P 21/02; C07K 2319/03; C07K 2319/32; C07K 2319/60

(57) ABSTRACT

Provided herein are linear expression constructs and methods of cell-free protein synthesis, optimised cell-free protein synthesis (CFPS) reagents, and methods for optimising CFPS reagents to increase protein expression yields. The constructs and methods are applicable to protein expression on a microfluidic device having hydrophobic surfaces. The constructs are applicable for making membrane or other hydrophobic proteins have multiple solubility tags.

### FIELD OF THE INVENTION

Provided herein are methods of providing nucleic acid expression constructs suitable for cell-free protein expression.

### BACKGROUND TO THE INVENTION

Protein expression requires a particular nucleic acid gene sequence along with reagents for synthesising the protein sequence based on the nucleic acid gene sequence. However the conditions required to express a particular protein are not obvious and must be determined empirically.

For cellular expression systems, there is a requirement for the expression vector to encode expression regulatory control elements matched to the host organism in which expression is being conducted (e.g. ribosome binding sites; codon usage; tRNA representation and structure; transcript modifications directing translation to the cytoplasm etc).

Cell-free protein synthesis (CFPS) regimes are attractive alternatives to cell-based expression systems as they can be treated as reagents rather than organisms, making them amenable to in vitro experimentation techniques. Additionally, cell-free systems are less sensitive to toxic protein synthesis; are open systems that can be modulated via addition of elements due to the lack of a cell membrane; are adaptable to high-throughput experiments; and can be used to good effect in small volumes. However, many of the cellular expression regulatory control paradigms still apply (e.g. incorrect ribosome binding motifs can lead to poor binding and poor transcription; incorrect codon usage can lead to inefficient translation etc).

Efficient protein synthesis relies on having the correct nucleic acid expression construct in the correct conditions. Protein synthesis and purification can be improved by attaching additional amino acids to the protein of interest, for example sequences improving solubility or tags for purification. In order to efficiently screen the optimal cell-free conditions for expression of a particular protein sequences it is desirable to provide a population of nucleic acid expression constructs. Furthermore, in order to identify the best DNA construct to generate a protein of interest it is desirable to provide a population of nucleic acid expression constructs. The invention herein describes methods for the preparation of nucleic acid constructs suitable for cell-free protein expression, and the use thereof.

Method for obtaining expression constructs include for example https://www.biotechrabbit.com/media/wysiwyg/files/btrproductinsert/RTS_Manuals/PIN-14008-002_RTS_Ecoli_LTGS_Histag_Manual.pdf. Disclosed herein are improved methods for making populations of linear expression constructs and obtaining proteins using these populations of linear expression constructs.

The expression constructs may be used for expressing membrane proteins by the attachment of suitable solubility tags. Integral membrane proteins (IMPs) account for nearly one third of all open reading frames in sequenced genomes and play vital roles in all cells including intra- and intercellular communication and molecular transport. Given their centrality in diverse cellular functions, IMPs have enormous significance in disease. However, understanding of this important class of proteins is hampered in part by a lack of generally applicable methods for overexpression and purification, two critical steps that typically precede functional and structural analysis. Most IMPs are naturally of low abundance and must be overproduced using recombinant systems. However, the yields of chemically and conformationally homogenous, active protein following overexpression in bacteria, yeast, insect cells or cell-free systems are often still too low to support functional and/or structural characterization, and can be further confounded by aggregation and precipitation issues. This limitation can sometimes be overcome using protein engineering whereby fusion partners are used to increase expression and promote membrane integration. Alternatively, mutations can be introduced to the IMP itself that enhance its stability or even render it water soluble. However, these approaches are largely trial and error, and the identification of suitable fusion partners or stabilizing mutations is neither trivial nor generalizable. Even when appropriate yields can be obtained, the hydrophobic nature of IMPs requires their solubilization in an active form, which is achieved mainly through the use of detergents that strip the protein from its native lipid environment and provide a lipophilic niche inside a detergent micelle. Because IMPs interact uniquely with each detergent, identifying the best detergents often involves lengthy and costly trials. A number of detergent-like amphiphiles have been developed that stabilize IMPs in solution including protein-based nanodiscs, peptide-based detergents, Styrene maleic-acid lipid particles (SMALPs) etc, and while these have helped to increase knowledge of IMPs, each type of amphiphile has its own limitations, and no universal reagent has been developed for wide use with structurally diverse IMPs.

### SUMMARY

The inventors have identified a need to rapidly generate nucleic acid constructs that are suitable for use in cell-free expression systems to produce target proteins or truncations thereof. They have therefore developed a method for rapidly installing the necessary regulatory and auxiliary components to a nucleic acid sequence that encodes a protein of interest, but which lacks the necessary regulatory and auxiliary elements which enable protein expression.

Furthermore, the method devised by the inventors enables the generation of constructs encoding a plurality of protein sequences from an initial nucleic acid sequence encoding for a single protein sequence or truncations thereof by the installation of fusion elements during the installation of the regulatory and auxiliary elements. For example, a single protein of interest can be expanded into 96 cell-free ready nucleic acid constructs that have different truncations, selections and positions of fusion proteins, purification tags, detection tags, cleavage sites, and linker sequences.

The approach described is particularly suited to CFPS rather than cell-based expression.

Unlike cell-based systems, in CFPS there is no amplification of the DNA expression construct. This means the multiplex population ratio is stable in CFPS but potentially changeable in cell-based systems depending on amplification efficiency. Thus the multiplex expression template population described herein is particularly suited for screening cell-free protein synthesis in a variety of conditions at the same time.

In one embodiment of the method devised by the inventors, a starting nucleic acid sequence—origination from a natural source (such as a cellular lysate or cDNA pool) or produced by de novo nucleic acid synthesis (chemical or enzymatic)—may be prepared for conversion into a cell-free ready construct by installation of adapter priming sequences. These priming sequences may be installed at 5′ and 3′ end of a nucleic acid sequence coding for a protein of interest. Alternatively, these priming sequences may be installed at (i) an internal sequence and 3′ end, (ii) 5′ end and an internal sequence, or (iii) two internal sequences, to generate length variants (i.e. N-terminal truncations, C-terminal truncations, or N- and C-terminal truncations) of the protein of interest. The inventors have identified a need to screen the expression characteristics of a plurality of expression constructs in a plurality of different lysates. They have therefore developed a universal expression cassette mix that is agnostic to these host-specific controls and lysate conditions, yet allows the efficient expression of any protein of interest in any lysate.

Whilst transcription of most genes can be controlled by the ubiquitous T7 promoter, translation is ribosome-specific and so requires a cell-specific 5′ untranslated region (5′UTR) or ribosome binding site for efficient translation. Unless the lysate and 5′UTR are matched, the yield and rate of protein expression is negatively impacted. It is therefore desirable to monitor expression using a variety of nucleic acid sequences with different ribosome binding sites in a variety of different lysates or assembled systems in order to optimize conditions for expression.

In order to simplify the sample preparation process and minimise the number, and type, of constructs required for a protein expression screen, it is attractive to use a “universal expression cassette” i.e. one that works equivalently well in all cell-free expression systems, regardless of origin species. Commercial expression cassettes exist that solve this problem by encoding a plurality of 5′UTR type in series, one after the other, within the same singular construct. However use of such a serial cassette means that an expressed protein contains significant amounts of unwanted amino acid sequence from the multiple UTR domains.

This invention solves the same problem but in an orthogonal manner: by constructing a multiplex expression cassette for a given protein of interest, where the multiplex expression cassette is a pool of expression cassette molecules that each encode single ribosome binding site (RBS) motifs. Each molecule of the multiplex expression cassette contains a single 5′UTR per strand, rather than a serial string of UTR's, the identity of 5′UTR is one of a number within the same pool. This means that when the universal expression cassette is used to “adapt” a given protein of interest coding sequence (CDS) the flanking regions of every molecule in the pool are identical in every regard except the sequence corresponding to the plurality of 5′UTR types. When a multiplex expression construct is supplied to any expression system of choice, transcription occurs from any cassette type, but subsequent translation only occurs from the subset of molecules whose 5′UTR matches the expression system.

This way, the same multiplex expression construct pool of varying UTR's can be used to express the same protein of interest in a variety of expression systems with optimal efficiency.

The advantage of this multiplex universal expression construct mix approach is that it delivers the benefit of a single linear expression construct (LEC)-lysate matched system (optimal ribosome binding site for efficient translation) without the drawbacks of the single LEC encoding multiple RBS in series (ribosomes binding on the outermost transcript RBS will be destabilised by the presence of the additional RBS on the same transcript in the intervening region between it and the start codon). So regardless which lysate type is used, the same mix should support efficient transcription/translation as it will work off the subset of templates within the pool that is optimal or the particular lysate.

Disclosed herein is a method of providing a nucleic acid expression construct suitable for cell-free protein expression, wherein the method comprises:
i. taking a double-stranded target nucleic acid sequence having ends A0 and B0;
ii. amplifying the double-stranded target nucleic acid with a left flank primer and a right flank primer wherein:
   the left flank primer comprises at least a promoter sequence, a sequence encoding for a ribosome binding site and, at its 3′ end, a sequence complementary to A0;
   and the right flank primer comprises a terminator sequence, a sequence encoding for a stop codon and, at its 3′ end, a sequence complementary to B0;
to produce a double-stranded expression construct suitable for cell-free protein expression.

Disclosed herein is a method of providing a nucleic acid expression construct suitable for cell-free protein expression, wherein the method comprises:
i. amplifying a starting nucleic acid sequence with a forward adapter primer and a reverse adapter primer wherein:
   the forward adapter primer comprises at its 3′ end a matching sequence A1 which can bind to a first region of the nucleic acid sequence, and at its 5′ end a sequence A0;
   and the reverse adapter primer comprises at its 3′ end a matching sequence B1 which can bind to a second region of the nucleic acid sequence, and at its 5′ end a sequence B0;
to produce a double-stranded target nucleic acid sequence having ends A0 and B0;
ii. amplifying the double-stranded target nucleic acid with a left flank primer and a right flank primer wherein:
   the left flank primer comprises at least a promoter sequence, a sequence encoding for a ribosome binding site and, at its 3′ end, a sequence complementary to A0;
   and the right flank primer comprises a terminator sequence, a sequence encoding for a stop codon and, at its 3′ end, a sequence complementary to B0;
to produce a double-stranded expression construct suitable for cell-free protein expression.

Disclosed herein is a method of providing a variety of nucleic acid expression constructs suitable for cell-free protein expression, wherein the method comprises:
i. taking a target nucleic acid sequence having ends A0 and B0, wherein A0 and/or B0 encode for protease cleavage sites in an expressed amino acid sequence;
ii. amplifying the target nucleic acid with multiple left flank primers and a single right flank primer to produce a population of constructs having different solubility tags, wherein:
   each left flank primer comprises at least a promoter sequence, a sequence encoding for a ribosome binding site, a solubility tag and, at its 3′ end, a sequence complementary to A0;
   and the right flank primer comprises a terminator sequence, a sequence encoding for a stop codon and, at its 3′ end, a sequence complementary to B0;
to produce a population of linear double-stranded expression constructs having different solubility tags suitable for cell-free protein expression.

Disclosed is a method of providing a variety of nucleic acid expression constructs suitable for cell-free protein expression, wherein the method comprises:
i. taking a double stranded target nucleic acid sequence having ends A0 and B0;
ii. amplifying the target nucleic acid with multiple left flank primers and one or more right flank primers to produce a population of constructs having different solubility tags or ribosome binding sites, wherein:
   each left flank primer comprises at least a promoter sequence, a sequence encoding for a ribosome binding site for a particular species, an optional solubility tag and, at its 3′ end, a sequence complementary to A0;
   and the right flank primer comprises a detection tag, an optional solubility tag, a terminator sequence, a sequence encoding for a stop codon and, at its 3′ end, a sequence complementary to B0;
   to produce a population of linear double-stranded expression constructs having a variety of solubility tags or ribosome binding sites suitable for cell-free protein expression of proteins which can be detected.

Disclosed is a method of providing a variety of nucleic acid expression constructs suitable for cell-free protein expression, wherein the method comprises:
i. taking one or more double stranded target nucleic acids, one of the nucleic acids having an end A0 and one having an end B0, wherein A0 and B0 are either connected directly in a single double stranded sequence or can be connected via hybridisation of multiple strands;
ii. amplifying the target nucleic acid with multiple left flank primers and one or more right flank primers to produce a population of constructs having different solubility tags or ribosome binding sites, wherein:
   each left flank primer comprises at least a promoter sequence, a sequence encoding for a ribosome binding site for a particular species, an optional solubility tag and, at its 3′ end, a sequence complementary to A0;
   and the right flank primer comprises a detection tag, an optional solubility tag, a terminator sequence, a sequence encoding for a stop codon and, at its 3′ end, a sequence complementary to B0;
iii. amplifying the products produced having the left and right flanks using amplification primers complementary to the left and right flanks to selectively amplify the full-length constructs and reduce the proportion of residual left flank primers, wherein the amplification uses at least 100 fold concentration of amplification primers in proportion to the flanking primers;
   to produce a population of linear double-stranded expression constructs having a variety of solubility tags or ribosome binding sites suitable for cell-free protein expression of proteins which can be detected.

The reaction can be performed in a single amplification, which can introduce ends A0 and B0 in a single amplification also using the left and right flank primers and the terminal amplification primers to produce the nucleic acid expression constructs.

A population of constructs having different ribosome binding sites can be prepared, either by making the amplicons separately and pooling the products, or by a single amplification using a mixture of left flank primers. The left flank primers are typically longer than 200 nucleotides in length. The left flank primers can be longer than 500 nucleotides in length. The left flank primers can be longer than 1000 nucleotides in length. The left flank primers can each contain one or more sequences expressing solubility tags, thereby allowing rapid screening of the best solubility tags after expression. The presence of protease cleavage sites allows the removal of the solubility tags if desired.

Also disclosed herein is an expression construct or population of expression constructs prepared according to the method described above.

Disclosed herein is a method of expressing a protein using a construct or population of constructs. The protein may be expressed using a cell-free system. The cell-free system may be a cell lysate. The cell-free system can be assembled from constituent components. The cell-free system can be assembled from purified recombinant elements. The cell-free system may be a blend of cell lysate and additional purified proteins.

Disclosed herein is a kit comprising an expression construct or population of expression constructs and components for cell-free protein expression.

Also disclosed herein is a kit comprising a population of left flank primers and a single right flank primer for amplification of a nucleic acid wherein:
i. the left flank primers each comprise a promoter sequence, a sequence encoding for a single ribosome binding site and, at its 3′ end, a sequence complementary to a nucleic acid to be amplified, wherein the population contains different ribosome binding sites; and
ii. the right flank primer comprises a terminator sequence, a sequence encoding for a stop codon and, at its 3′ end, a sequence complementary to a nucleic acid to be amplified; and
wherein the left and right flank primers are independently between 200 and 3000 nucleotides in length.

Also disclosed herein is a kit comprising a population of left flank primers and a single right flank primer for amplification of a nucleic acid wherein:
i. the left flank primers each comprise a promoter sequence, a sequence encoding for a ribosome binding site and one or more solubility tags, and at its 3′ end a sequence complementary to a nucleic acid to be amplified, wherein the population contains different solubility tags; and
ii. the right flank primer comprises a sequence coding for a detection tag, a sequence coding for a purification tag, a sequence encoding for a stop codon and, at its 3′ end, a sequence complementary to a nucleic acid to be amplified.

The left flank primer may end with the A0 complementary sequence 5′-CTCGAGGTTCTGTTCCAAGGACCT-3′.

The right flank primer ends with the B0 complementary sequence 5′-GAGAACCTGTACTTCCAGAGC-3′.

Each of the left and right flank primers may be produced by amplification. The left and right flank primers may be used in single stranded or double stranded form.

### Cassette Mixes

Generally a set (>2) of left flank (LF) primers are manufactured independently. The primers are larger than the primers used in standard amplification reactions, and are referred to as megaprimers. For a mixture of expression cassettes, these megaprimers are identical in every regard except in the nature of the RBS sequence they encode. One RBS might be optimal for E coli expression systems, a second compatible with mammalian expression systems (e.g. EMCV), a third compatible with plant expression systems (e.g. TMV), a fourth agnostic to any specific expression system (e.g. species-independent translation system, SITS). Each left flank megaprimer can be longer than 500 nucleotides in length. Each left flank megaprimer can be longer than 1000 nucleotides in length.

Purified LF megaprimers described above are pooled together in a molar ratio determined empirically to form a multiplex LF megaprimer pool.

A single right flank (RF) megaprimer (downstream from the CDS, without the expression control elements) is added to the multiplex forward megaprimer pool to make the final multiplex megaprimer pool.

The multiplex megaprimer pool is combined with a template molecule (typically the coding sequence of a protein of interest flanked by adapter sequences compatible with the LF and RF megaprimers).

PCR reagents are added (DNA polymerase, dNTPs, buffer) to the mix and the reaction is amplified for a number of cycles, in order to add the flanking LF and RF megaprimer arms to the template, thereby generating the Universal multiplex expression construct pool.

This Universal multiplex expression construct pool is ready to be used as the DNA expression construct input for a CFPS reaction. As the pool contains a mix of molecules with different RBS coding sequences, the same pool is expressible in a plurality of different CFPS lysates using at least one of the available constructs

Whilst this approach has been developed to interface with cell-free expression systems, the concept of a universal multiplex expression cassette could equally be applied to cell-based systems. In these cases, a multiplex mix of plasmid expression constructs can be envisaged which when transformed would give rise to a population of cells, each containing a plasmid whose RBS is different. Cells transformed with an inappropriate RBS will be selected against during cell growth leading to enrichment of the appropriate cell: RBS combination.

The expressed protein may be fused to a peptide detection tag. The detection tag may be one component of a fluorescent protein, which can be detected by binding to a further polypeptide being a complementary portion of the fluorescent protein. The fluorescent protein could include sfGFP, GFP, eGFP, ccGFP, deGFP, frGFP, eYFP, eBFP, eCFP, Citrine, Venus, Cerulean, Dronpa, DsRED, mKate, mCherry, mRFP, FAST, SmURFP, miRFP670nano. For example the peptide tag may be GFP₁₁ and the further polypeptide GFP₁₋₁₀. The peptide tag may be one component of sfCherry. The peptide tag may be sfCherry₁₁ and the further polypeptide sfCherry₁₋₁₀. The peptide tag may be CFAST₁₁ or CFAST₁₀ and the further polypeptide NFAST in the presence of a hydroxybenzylidene rhodanine analog. The peptide tag may be ccGFP₁₁ and the further polypeptide ccGFP₁₋₁₀.

The complementary GFP₁₁ peptide amino acid sequence could be the following:

1. KRDHMVLLEFVTAAGITGT
2. KRDHMVLHEFVTAAGITGT
3. KRDHMVLHESVNAAGIT
4. RDHMVLHEYVNAAGIT
5. GDAVQIQEHAVAKYFTV
6. GDTVQLQEHAVAKYFTV
7. GETIQLQEHAVAKYFTE

or a truncated version thereof. Truncations may involve a shortening of up to 5 amino acids from the N terminus, the C terminus or a combination thereof.

The detection tag may also be one component of a protein that forms a detectable substrate, such as a luminescent or colorigenic substrate. The protein could include beta-galactosidase, beta-lactamase, or luciferase.

The protein may be fused to multiple tags. For example the protein may be fused to multiple GFP₁₁ peptide tags and the synthesis occurs in the presence of multiple GFP₁₋₁₀ polypeptides. For example the protein may be fused to multiple sfCherry₁₁ peptide tags and the synthesis occurs in the presence of multiple sfCherry₁₋₁₀ polypeptides. The protein of interest may be fused to one or more sfCherry₁₁ peptide tags and one or more GFP₁₁ peptide tags and the synthesis occurs in the presence of one or more GFP₁₋₁₀ polypeptides and one or more sfCherry₁₋₁₀ polypeptides.

Any protein of interest may be synthesised. The protein may be an enzyme, for example a terminal deoxynucleotidyl transferase (TdT) enzyme or a truncated version thereof or the homologous amino acid sequence of a terminal deoxynucleotidyl transferase (TdT) enzyme in other species or the homologous amino acid sequence of Polμ, Polβ, Polλ, and Polθ of any species or the homologous amino acid sequence of X family polymerases of any species.

The protein of interest may be a membrane protein or similar hydrophobic protein. This approach may be used to solubilize not only membrane proteins but also intrinsically disordered proteins or any proteins that readily unfold to expose their hydrophobic core causing aggregation. The solubility tag or decoy/shield proteins may cover up hydrophobic regions that cause soluble proteins to aggregate. The protein may be stabilized by attachment to multiple solubility tags, for example tags at both the C and N sides of the trans-membrane domain. The protein may include an amphipathic shield domain protein moiety which can act as a solubility tag; an integral membrane protein moiety; and a water soluble expression decoy protein moiety. The amphipathic shield protein moiety may be coupled to the integral membrane protein moiety's C-terminal domain and the water soluble expression decoy protein moiety coupled to the integral membrane protein moiety's N-terminal domain. The amphipathic shield protein moiety may be coupled to the integral membrane protein moiety's N-terminal domain and the water soluble expression decoy protein moiety coupled to the integral membrane protein moiety's C-terminal domain. Thus the hydrophobic protein is provided with hydrophilic solubility tags at both the N and C terminus in the form of shield and decoy proteins such as lipoproteins, for example apoliproteins such as APoE.

### BRIEF DESCRIPTION OF THE DRAWINGS

FIG. 1: A schematic outlining the process of preparing an expression cassette using a two-stage amplification process. The first stage introduces universal sequences (A0 and B0). In the example shown the sequences code for protease cleavage sites such as TEV and 3C. The amplification gives a double stranded target amplicon having ends A0 and B0. This target amplicon can be further amplified using the flanking megaprimers, the megaprimers having sequences which hybridise to A0 and B0, to install regulatory elements and optionally fusion peptide/protein sequences.

FIG. 2: Lysate-specific expression constructs. The natural process for generating lysate constructs involves separate expression in separate systems. The nature of the lysate means the correct binding site (RBS) is chosen. There is no combining of different binding sites as the lysate is known,

FIG. 3: Universal expression construct with multiple RBS in series in a single construct molecule as seen in the art. The expressed protein contains the sequence of multiple UTRs, depending on which RBS initiates expression.

FIG. 4: The method of the invention; multiplex universal expression construct comprising a plurality of different expression cassettes each harboring only a single lysate-specific RBS.

FIG. 5: The method of the invention; multiplex universal expression constructs comprising a plurality of different expression cassettes each harboring only a single lysate-specific RBS. Each expression construct is synthesized separately and pooled following synthesis. Expression constructs can be present in an inefficient lysate, acting merely as spectator molecules during the expression using the efficient system.

FIG. 6: Schematic outlining the Universal multiplex expression construct pool synthesis process.

FIG. 7; Preparation of a population of expression constructs having a series of truncations. FIG. 7a shows a selection of primers having sequences A0′, A0″, A0′″ hybridizing to various positions in a gene of interest. The first amplification stage introduces universal sequences (A0 and B0) onto a series of truncations of different length defined by where A0′, A0″, A0′″ hybridise. The amplification gives a selection of different length double stranded target amplicons having ends A0 and B0. FIG. 7b; These target amplicons can be further amplified using the flanking megaprimers, the megaprimers having sequences which hybridise to A0 and B0, to install regulatory elements and optionally fusion peptide/protein sequences. Thus a population of constructs having truncations of the gene of interest can be prepared.

FIG. 8: A standardized “mastermix reagent”. The mastermix makes the manufacture of universal expression constructs very simple. In order to make robust, the megaprimers are supplemented with single stranded terminal primers at a much higher concentration to enrich for the full-length amplicons. This way, the megaprimers provide the specificity (i.e. enable a functional construct to be generated) but the inclusion of the terminal primers allows the number of moles of amplicon to be dramatically increased (compared to if they are not present in the mix).

FIG. 9: An exemplary 12 construct library. Each protein of interest is flanked by a variety of optional solubility tags, purification tags, detections tags, buffer sequences, promoter sequences and binding sites, either on the C or N terminus of the expressed protein. The library mix can be screened in parallel to determine the optimal conditions for protein expression and isolation.

FIG. 10: 1% TBE agarose gel AdaptPCR amplicons (30 cycle)

FIG. 11: 1% TBE agarose gel UMA-LEC amplicons (30 cycle).

FIG. 12: Calibrated CFPS expression data for UMA-LEC constructs in LS70 (1 nM, 18 hrs)

FIG. 13: An exemplary schematic showing a multi-part assembly to make long nucleic acid constructs by amplification.

FIG. 14: Production of a 210 kDa Cas9 protein from a 5 kb construct

FIG. 15: Production of a 310 kDa Acetyl COA carboxylase from a 8 kb base pair construct.

FIG. 16: Activity assay for purified Cas9. The same amount of target DNA is used per reaction (100 ng). Cas9 dilution series shown. Cleaved products have expected molecular weight. Cas9 shows DNA digestion activity. At the highest concentration (3000 ng) excess Cas9 causes aggression of DNA target, resulting in no cleavage.

FIG. 17: Activity assay for purified Cas9. Cas9 optimal cleavage efficiency at 700 ng (1:7 target:enzyme.

FIG. 18: Fluorescent gel images of expressed proteins for two nucleic acid inserts (oid 51 and oid 246). More PCR cycles gives in increase in shortened proteins.

FIG. 19: Varying ratio of input primers and template concentrations for PCR conditions.

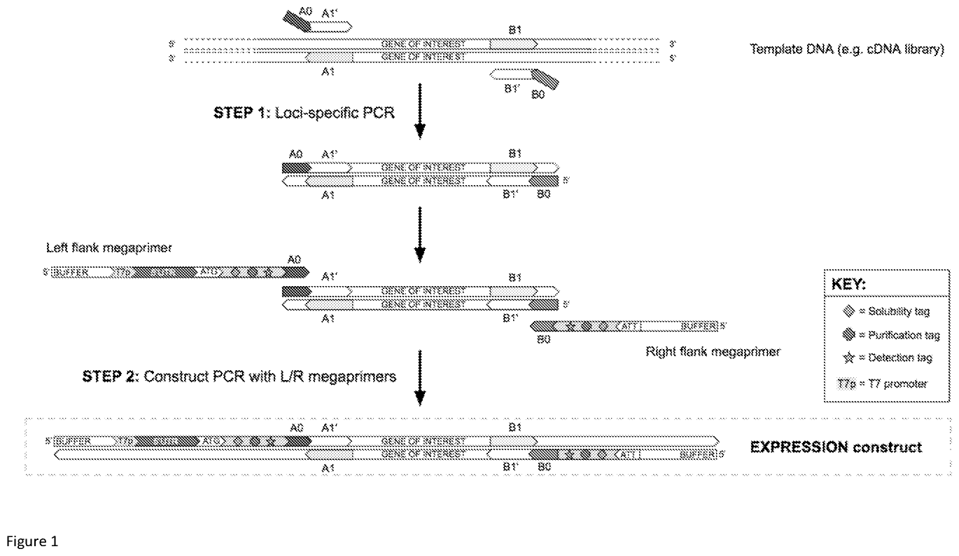

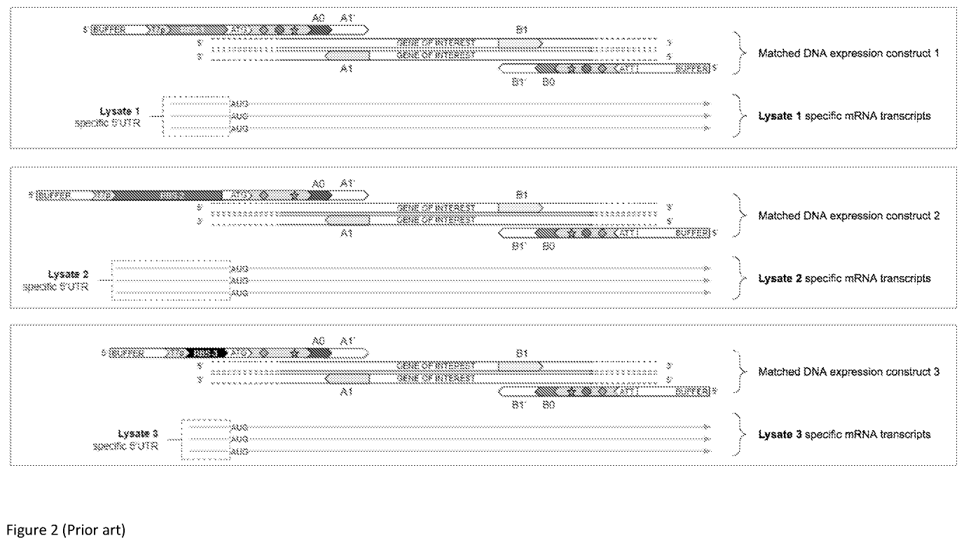

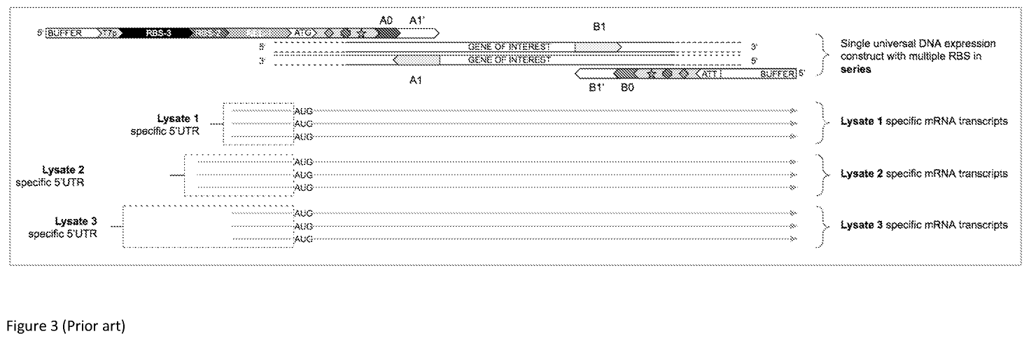

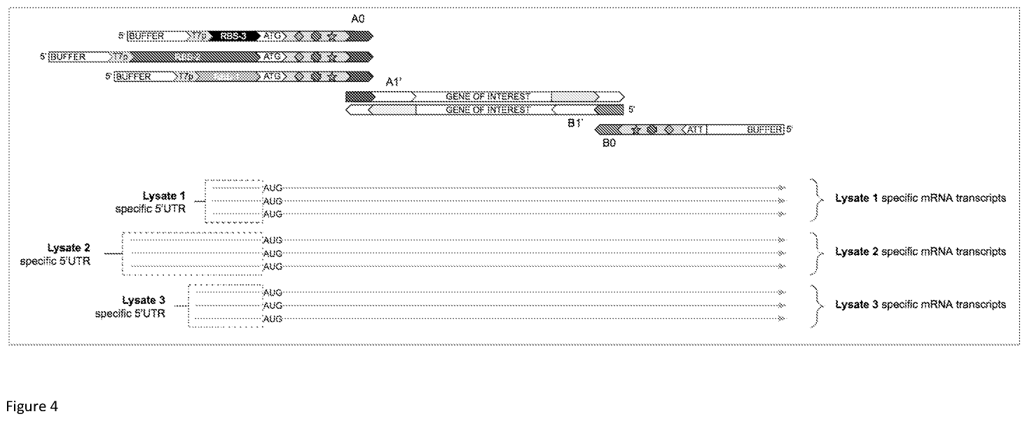

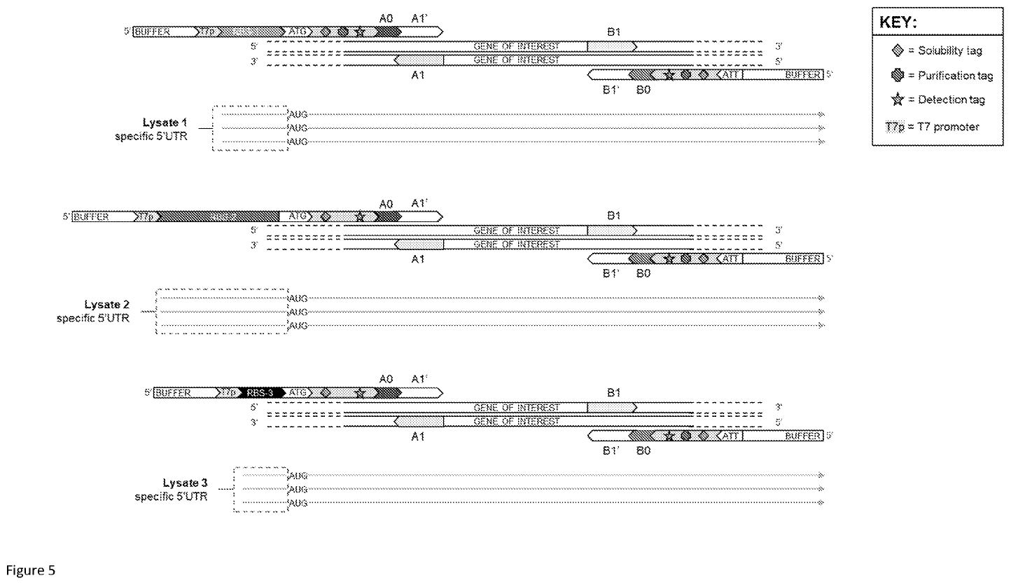

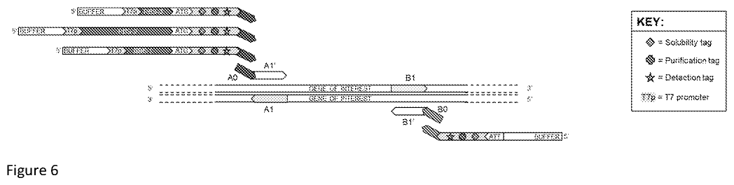

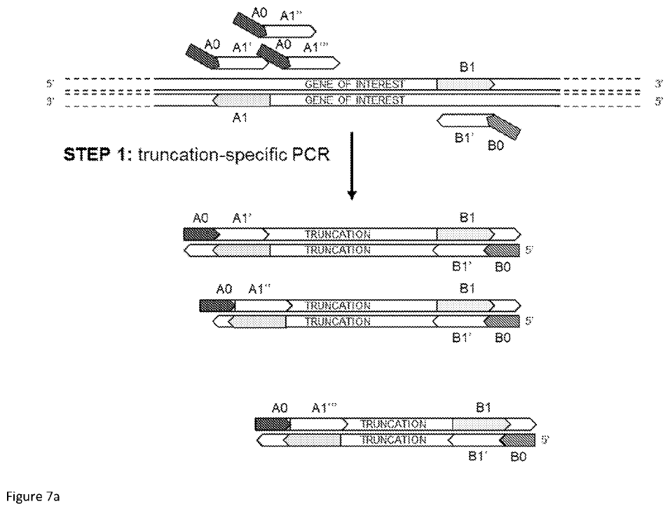

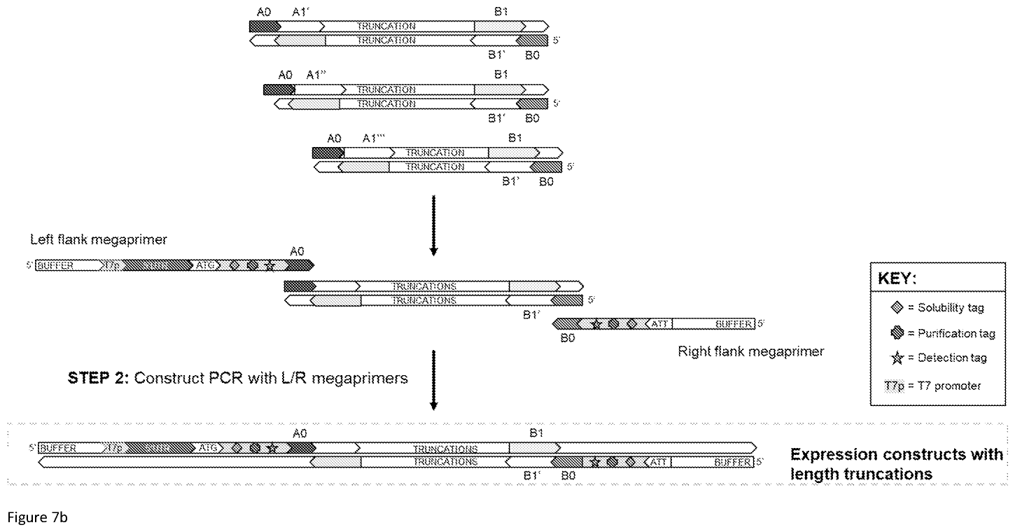

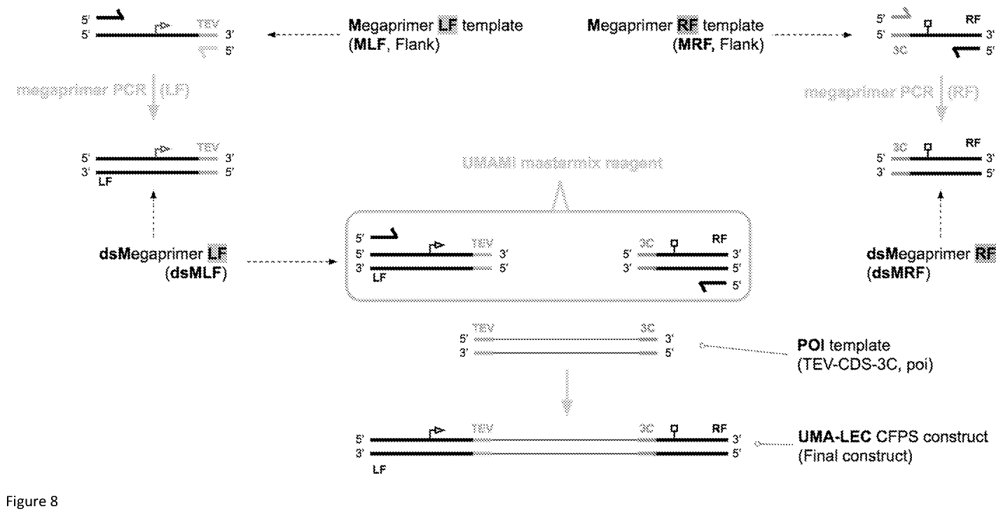

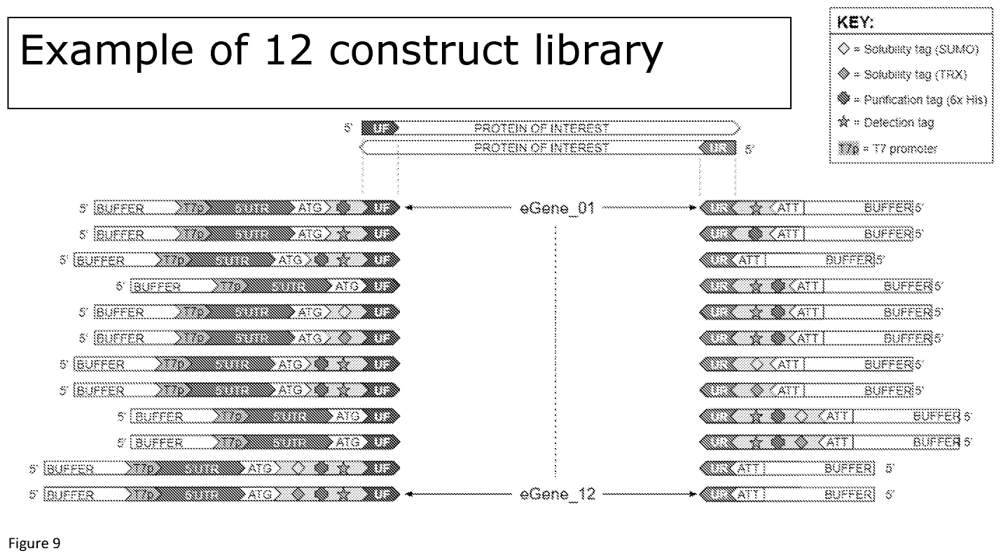

### DETAILED DESCRIPTION OF THE INVENTION

### Definitions

Target nucleic acid sequence=the sequence coding for a protein that already has priming sequences.
Priming sequence=the sequence (for example A0/B0) which the left/right flank primers will bind to.
Left/right flank primer=primers that will install the left and right flanks (long sequences) of the construct to enable protein expression.
Starting nucleic acid sequence=a sequence from which a target nucleic acid sequence can be generated by appending priming sequences (e.g. installing A0/B0)
Adapter priming sequence=the variable loci sequence (A1/B1) in the starting nucleic acid sequence which the forward/reverse adapter primers will bind to.

The terms ‘left’ and right’ are used herein to symbolizing opposing ends of a template, and could equally be marked as ‘end 1’ and ‘end 2’ or ‘start codon flank’ and ‘stop codon flank’. The term left and right have no positional meaning and are used to aid interpretation of the claims in relation to diagrams. The left flank and right flank elements could be transposed without affecting the meaning of the terms (for example the right flank could have a start codon and the left flank a stop codon).

The terms A0, A1 etc are used to signify regions of nucleic acid sequence, and apply equally to the complementary sequences A1′ and A0′ which hybridise thereto. A1 and A1′ are loci specific sequences. A0 and B0 are universal sequences.

Thus the flow can be envisaged as:
Starting sequence (biological sample)→Target sequence (short adapters attached having known priming sequences)→Construct suitable for CFPS (long flanks attached). The primer sequences A0 and B0 are attached to starting sequences to make target sequences. The target sequences are amplified using primers specific to A0 and B0.

Priming sequences A0/B0 enable left/right flank primers to bind and install left/right flanks. The priming sequences can include a sequence coding for a protease cleavage site.

Adapter priming sequences A1/B1 enable forward/reverse adapter primers to bind and install priming sequences A0/B0 in the amplified target. A1 and B1 are ‘loci specific’ and vary depending on the starting nucleic acid.

The amplification can be done in a single step having multiple primers. Thus primers A1/A0 and B1/B0 can be used in a composition with the left and right flank primers and the amplification primers to obtain the constructs ready for CFPS.

Disclosed herein is a method of providing a nucleic acid expression construct suitable for cell-free protein expression, wherein the method comprises:
i. taking a target nucleic acid having ends A0 and B0;
ii. amplifying the target nucleic acid with a left flank primer and a right flank primer wherein:
   the left flank primer comprises at least a promoter sequence, a sequence encoding for a ribosome binding site and, at its 3′ end, a sequence complementary to A0;
   and the right flank primer comprises a terminator sequence, a sequence encoding for a stop codon and, at its 3′ end, a sequence complementary to B0;
to produce a double-stranded expression construct suitable for cell-free protein expression.

Disclosed herein is a method of providing a nucleic acid expression construct suitable for cell-free protein expression, wherein the method comprises:
i. amplifying a starting nucleic acid sequence with a forward adapter primer and a reverse adapter primer wherein:
   the forward adapter primer comprises at its 3′ end a matching sequence A1 which can bind to a first region of the nucleic acid sequence, and at its 5′ end a sequence A0;
   and the reverse adapter primer comprises at its 3′ end a matching sequence B1 which can bind to a second region of the nucleic acid sequence, and at its 5′ end a sequence B0;
to produce a double-stranded target nucleic acid having ends A0 and B0;
ii. amplifying the double-stranded target nucleic acid with a left flank primer and a right flank primer wherein:
   the left flank primer comprises at least a promoter sequence, a sequence encoding for a ribosome binding site and, at its 3′ end, a sequence complementary to A0;
   and the right flank primer comprises a terminator sequence, a sequence encoding for a stop codon and, at its 3′ end, a sequence complementary to B0;
to produce a double-stranded expression construct suitable for cell-free protein expression.

Also disclosed herein is an expression construct or population of expression constructs prepared according the method described above.

The matching sequences A1 and B1 can independently between 6 and 100 nucleotides, more preferably 10 and 50 nucleotides. These matching sequences may or may not be fully complementary. Depending on whether the input amplicon is double or single stranded, the primers may be complementary to the sense or antisense strands. Where the template used is ssDNA, the one primer would only be complementary once the first copy of the template strand was made. Thus one primer is complementary to and hybridises to one strand and one primer hybridises to the complementary strand.

The method may use one or more internally complementary regions to allow extensions from two shorter extension products. Thus a multi-part assembly may be performed in order to produce longer nucleic acid constructs. Thus a single amplification can be used to produce nucleic acid constructs of for example greater than 3 kb. The nucleic acid construct may be 3-10 kb.

The method may use a two part assembly where a first nucleic acid has end A0 and a second nucleic acid end B0. The strands are complementary, allowing extension against each other. The ends can have regions C1 and C1′.

The method may use a nucleic acid having an end A0 and an end C1, and a separate nucleic acid having an end B0 and end C1′, wherein C1 and C1′ are complementary, to produce a multi-part extension product having A0 and B0 using two shorter extension products. This reaction can be performed as part of an extension using the flank primers and amplification primers. In such a case, the template may not have ‘ends’ B0 and A0, as the sequences may be internal in some of the templates. In such case A0 and B0 are connected via hybridisation.

The method may use a three part assembly using a first nucleic acid having end A0 and a second nucleic acid having end B0, plus a third strand which can link A0 and B0 via hybridisation. The strand ends are are complementary, allowing extension against each other. The ends can have regions C1 and C1′ and D1 and D1′ etc. Such splint assemblies can use multiple parts as needed to produce the desired length templates.

Sequences A0 and B0 can encode for protease cleavage sites in an expressed amino acid sequence. The protease can be a cysteine, serine, or threonine protease, an aspartic protease, glutamic protease or metallo protease. Encoding protease cleavage sites enables the cleavage of fusion elements added via the method of the invention to be cleaved in situ or downstream to yield the original protein of interest.

The protease can be selected from the following: TEV, C3, enterokinase (EK) light chain, factor Xa (FXA), furin (FN) or thrombin. Enterokinase (EK) cleaves a NNNNL motif. Factor Xa cleaves a I(E/D)GR motif. Furin cleaves a RXXR motif. Thrombin cleaves a LVPRGS motif. TEV Protease is a cysteine protease that recognizes the sequence Glu-Asn-Leu-Tyr-Phe-Gln-(Gly/Ser) and cleaves between the Gln and Gly/Ser residues. C3 Protease is a cysteine protease that recognizes Leu-Glu-Val-Leu-Phe-Gln/Gly-Pro (LEVLFQ/GP) and cleavage occurs between the Gln and Gly-Pro residues.

The primer sequences can include sequences:

5′-GAGAACCTGTACTTCCAGAGC-3′
(TEV cleavage sequence ENLYFQS)
5′-TCCTTGGAACAGAACCTCGAG-3′
(3′-5′ LEVLFQG 3C cleavage sequence)
5′-CTCGAGGTTCTGTTCCAAGGACCT-3′
(LEVLFQGP 3C cleavage sequence))

The left flank primer may further comprise a sequence or plurality of sequences encoding for ribosome interactions sites selected from alternative ribosome binding sites (RBS) or internal ribosome entry sites. The left flank or right flank primer may code for a selection of solubility tags. The left flank primer may end with the A0 complementary sequence 5′-CTCGAGGTTCTGTTCCAAGGACCT-3′. This sequence will express the amino acid sequence LEVLFQGP, a 3C protease cleavage sequence.

The left flank primer and/or the right flank primer may further comprise a DNA sequence or plurality of DNA sequences encoding for additional peptide structures selected from detection tags, purification tags, solubility tags, linkers and/or spacers.

The detection tags may be selected from a component part of a fluorescent protein.

Affinity tags may be appended to proteins so that they can be purified from their crude biological source using an affinity technique The purification tags may be selected from for example FLAG-tag, His-tag, GST-tag, MBP-tag, STREP-tag. The Flag® tag, also known as the DYKDDDDK-tag, is a popular protein tag that is commonly used in affinity chromatography and protein research. His tags are polyhistidine strings of amino acids, typically between 6 and 9 histidine amino acids in length.

The proteins may be membrane proteins or other proteins having intrinsically disordered regions or any proteins that readily unfold to expose their hydrophobic core causing aggregation. The proteins may have multiple solubility tags attached to ensure the membrane or hydrophobic protein is soluble in the absence of a membrane. Preparation of stabilised membrane proteins in described in U.S. Pat. No. 10,961,286, incorporated herein by reference in its entirety.

As used herein, the term “integral membrane protein” (IMP) includes a type of transmembrane protein held in the bilayer of a cellular membrane by lipid groups with tight binding to other proteins. The IMPs of the present invention play vital roles in all cells including intra- and intercellular communication and molecular transport. The IMPs of the present invention are uniquely stable and water soluble following extraction from their native environment (e.g., a cellular membrane) without the use of detergents and/or detergent-like amphiphiles, overproduction using recombinant systems, protein engineering, and/or mutations to the IMP itself, thereby allowing for improved functional and structural studies of IMPs as well as in vitro reconstitution of enzymatic activity or in vitro reconstitution of a biological pathway involving water soluble IMP enzymes and engineering of biological/metabolic pathways directly in living cells involving the water soluble IMPs.

The IMPs of the present invention may be selected from the group consisting of bitopic α-helical IMPs, polytopic α-helical IMPs, IMPs with multiple helices, and polytopic β-barrel IMPs. The IMPs of the present invention may be classified structurally as β-barrel or α-helical bundles. β-barrels may be expressed as inclusion bodies, purified and refolded for structural studies, whereas α-helical bundles are less likely to produce soluble active forms after refolding.

In one embodiment, the bitopic α-helical IMP is human cytochrome b5 (cyt b₅). Cyt b₅ is a 134-residue bitopic membrane protein consisting of six α-helices and five β-strands folded into three distinct domains: (i) an N-terminal haeme-containing soluble domain; (ii) a C-terminal membrane anchor; and (iii) a linker or hinge region that connects the two domains. Native cyt b₅ stimulates the 17,20-lyase activity of cytochrome P450c17 (17α-hydroxylase/17,20-lyase; CYP17A0). In particular, a molar equivalent of cyt b₅ increases the rate of the 17,20-lyase reaction 10-fold, via an allosteric mechanism that does not require electron transfer. Given that the C-terminal transmembrane helix of cyt b₅ is required to stimulate the 17,20-lyase activity of human CYP17A0, the ApoAl* shield may, in one embodiment, be sufficiently flexible to allow the protein-protein interactions that are necessary to promote proper function.

In another embodiment, the polytopic α-helical IMP is selected from the group consisting of Homo sapiens hydroxy steroid dehydrogenase (HSD173), H. sapiens glutamate receptor A2 (GluA2), E. coli DsbB (DsbB), H. sapiens Claudin1 (CLDN1), H. sapiens Claudin3 (CLDN3), H. sapiens sapiens steroid 5a-reductase type 1 (S5αR1), H. sapiens sapiens steroid 5a-reductase type 2 (S5αR2), and Halobacterium sp. NRC-1 bacteriorhodopsin (bR). In one embodiment, a small (110 amino acids) polytopic α-helical IMP from E. coli named ethidium multidrug resistance protein E (EmrE), comprised of four transmembrane α-helices having 18-22 residues per helix with very short extramembrane loops, may be used. EmrE as described herein is the archetypical member of the small multidrug resistance protein family in bacteria and confers host resistance to a wide assortment of toxic quaternary cation compounds by secondary active efflux.

In another embodiment, the polytopic β-barrel IMP is selected from the group consisting of E. coli OmpX (OmpX) and Rattus norvegicus voltage-dependent anion channel 1 (VDAC1).

In another embodiment, the IMPs with multiple helices may further include, for example, polytopic β-barrel membrane proteins such as outer membrane proteins including, for example, OmpX, OmpX^{a}, OmpA, OmpA^{a}, PagP^{a}, NspA, OmpT, OpcA, NalP, OmpLA, TolC, FadL, OmpF, PhoE, Porin, OmpK36, Omp32, MspA, LamB, Maltoporin, ScrY, BtuB, FhuA, FepA, and FecA. See Tamm et al., “Folding and Assembly of β-barrel Membrane Proteins,” Biochimica et Biophysica Acta 1666:250-263 (2004), which is hereby incorporated by reference in its entirety. Non-constitutive β-barrel membrane proteins include, but are not limited to, α-Hemolysin and LukF. See Tamm et al., “Folding and Assembly of β-barrel Membrane Proteins,” Biochimica et Biophysica Acta 1666:250-263 (2004), which is hereby incorporated by reference in its entirety.

In yet another embodiment, the IMP is selected from the group consisting of G protein-coupled receptors (GPCR) and olfactory receptors. GPCRs can include the Class A (Rhodopsin-like) GPCRs, which bind amines, peptides, hormone proteins, rhodopsin, olfactory prostanoid, nucleotide-like compounds, cannabinoids, platelet activating factor, gonadotropin-releasing hormone, thyrotropin-releasing hormone and secretagogue, melatonin and lysosphingolipid and LPA. GPCRs with amine ligands can include, without limitation, acetylcholine or muscarinic, adrenoceptors, dopamine, histamine, serotonin or octopamine receptors); peptide ligands include but are not limited to angiotensin, bombesin, bradykinin, anaphylatoxin, Fmet-leu-phe, interleukin-8, chemokine, cholecystokinin, endothelin, melanocortin, neuropeptide Y, neurotensin, opioid, somatostatin, tachykinin, thrombin vasopressin-like, galanin, proteinase activated, orexin and neuropeptide FF, adrenomedullin (G10D), GPR37/endothelin B-like, chemokine receptor-like and neuromedin U.

As used herein, the term “amphipathic shield domain protein” includes any protein that displays both hydrophilic and hydrophobic surfaces and is often associated with lipids as membrane anchors or involved in their transport as soluble particles. The amphipathic shield domain protein, in one embodiment, serves as a molecular shield to sequester large lipophilic surfaces of the IMP from water. Apolipoproteins are proteins that bind lipids (oil-soluble substances such as fats, cholesterol and fat soluble vitamins) to form lipoproteins.

They transport lipids in blood, cerebrospinal fluid and lymph. The lipid components of lipoproteins are insoluble in water. However, because of their detergent-like (amphipathic) properties, apolipoproteins and other amphipathic molecules (such as phospholipids) can surround the lipids, creating a lipoprotein particle that is itself water-soluble,

In various embodiments, the amphipathic shield domain protein may be selected from the group consisting of Apolipoprotein A (Apo-Al, Apo-A2, Apo-A4, and Apo-A5), apolipoprotein B (ApoB), apolipoprotein C (ApoC), apolipoprotein D (ApoD), apolipoprotein E (ApoE), apolipoprotein F (ApoF), apolipoprotein L (ApoL), apolipoprotein M (ApoM), apolipoprotein M (ApoM) and a peptide self-assembly mimic (PSAM). In particular, the amphipathic shield domain protein may be apolipoprotein A0 (ApoAl). As used herein, ApoAl avidly binds phospholipid molecules and organizes them into soluble bilayer structures or discs that readily accept cholesterol. ApoAl contains a globular amino-terminal (N-terminal) domain (residues 1-43) and a lipid-binding carboxyl-terminal (C-terminal) domain (residues 44-243). In one embodiment, the ApoAl may be truncated (ApoAl*). Truncated variants of ApoA0 include, but are not limited to, human ApoAl lacking its 43-residue globular N-terminal domain. As used herein, ApoA0 exhibits remarkable structural flexibility, and may adopt a molten globular-like state for lipid-free ApoAl under conditions that may allow it to adapt to the significant geometry changes of the lipids with which it interacts. The present invention designs chimeras in which, for example, ApoAl* may be genetically fused to the C terminus of an IMP target. Expression of these chimeras in the cytoplasm of Escherichia coli may yield appreciable amounts of globular, water-soluble IMPs that are stabilized in a hydrophobic environment and retain structurally relevant conformations. The approach provides, inter alia, a facile method for efficiently solubilizing structurally diverse IMPs, for example in both bacteria and human cells, as a prelude to functional and structural studies, all without the need for detergents or lipid reconstitutions. In one embodiment, a plasmid may be used which encodes a chimeric protein in which ApoAl is fused to the C-terminus of EmrE. In another embodiment, the amphipathic shield domain protein is a peptide self-assembly mimic (PSAM).

The shield domain may be made of multiple proteins with optional linkers. The shield may be multiple proteins selected from apolipoprotein A (ApoA), apolipoprotein B (ApoB), apolipoprotein C (ApoC), apolipoprotein D (ApoD), apolipoprotein E (ApoE), apolipoprotein H (ApoH), and a peptide self-assembly mimic (PSAM).

The solubility tag may take the form of a water soluble expression decoy protein. As used herein, the term “water soluble expression decoy protein” includes any protein which serves to direct an IMP into cellular cytoplasm. The water soluble expression decoy protein may assist in “tricking” a hydrophobic IMP into thinking that it is not hydrophobic. The desired water soluble decoy protein for a particular IMP can be identified by the methods described herein by producing a variety of nucleic acid sequences expressing a shield domain protein-IMP-variety of decoy conjugates and seeing which nucleic acid construct best expresses soluble and detectable protein, thereby identifying a preferred decoy conjugate. The decoy can be attached to the C or N terminus.

Disclosed is a method wherein the nucleic acid encodes a tripartite fusion protein, said nucleic acid molecule comprising:
a first nucleic acid moiety encoding one or more amphipathic shield domain protein(s) selected from the group consisting of apolipoprotein A (ApoA), apolipoprotein B (ApoB), apolipoprotein C (ApoC), apolipoprotein D (ApoD), apolipoprotein E (ApoE), apolipoprotein H (ApoH), and a peptide self-assembly mimic (PSAM);
a second nucleic acid moiety encoding an integral membrane protein; and
a third nucleic acid moiety encoding one or more solubility tag(s) in the form of a water soluble expression decoy protein.

The a first nucleic acid moiety encoding an amphipathic shield domain protein and the a second nucleic acid moiety encoding an integral membrane or hydrophobic protein may be located between regions A0 and B0, and become attached to a variety of solubility tags/decoy proteins using the methods described herein.

Disclosed is a method wherein the nucleic acid encodes a tripartite fusion protein, said nucleic acid molecule comprising:
a first nucleic acid moiety encoding an amphipathic shield domain protein selected from the group consisting of apolipoprotein A (ApoA), apolipoprotein B (ApoB), apolipoprotein C (ApoC), apolipoprotein D (ApoD), apolipoprotein E (ApoE), apolipoprotein H (ApoH), and a peptide self-assembly mimic (PSAM);
a second nucleic acid moiety encoding an integral membrane protein; and
a third nucleic acid moiety encoding a solubility tag in the form of a water soluble expression decoy protein, wherein said first nucleic acid moiety is coupled to said second nucleic acid moiety's 3′ end and said third nucleic acid moiety is coupled to said second nucleic acid moiety's 5′ end, said coupling being direct or indirect.

The right flank primers can include a variety of solubility tags for screening the expression and solubility of the integral membrane protein via a selection of water soluble expression decoy proteins.

The shield and/or decoy proteins may be connected to the membrane protein via a cleavable linker such as a sequence cleavable using a protease. The protease may be present as an additive during the expression process in order to cleave the shield or decoy proteins from the membrane proteins.

Where present, the binding moiety for purification may contain four or more amino acids. The binding sequences may contain 4-30 amino acids. The binding moiety may be selected from:

Alfa-tag (SRLEEELRRRLTE)
Avi-tag (GLNDIFEAQKIEWHE)
C-tag (EPEA)
Calmodulin-tag (KRRWKKNFIAVSAANRFKKISSSGAL)
Dogtag (DIPATYEFTDGKHYITNEPIPPK)
E-tag (GAPVPYPDPLEPR)
FLAG (DYKDDDDK)
G4T (EELLSKNYHLENEVARLKK)
HA (YPYDVPDYA)
His (HHHHHH)
Isopeptag (TDKDMTITFTNKKDAE)
lanthanide binding tag (LBT) (FIDTNNDGWIEGDELLLEEG)
Myc (EQKLISEEDL)
NE-Tag (TKENPRSNQEESYDDNES)
Poly Glutamate-tag (EEEEEEE)
Poly Arginine-tag (RRRRRRR)
Rho1D4-tag (TETSQVAPA)
SBP-tag (MDEKTTGWRGGHVVEGLAGELEQLRARLEHHPQGQREP)
Sdytag (DPIVMIDNDKPIT)
SH3 (STVPVAPPRRRRG)
SNAC (GSHHW)
Snooptag (KLGDIEFIKVNK)
Softag 1 (SLAELLNAGLGGS)
Softag 3 (TQDPSRVG)
Spot-tag (PDRVRAVSHWSS)
Spytag (AHIVMVDAYKPTK)
S-tag (KETAAAKFERQHMDS)
Strep-tag (AWAHPQPGG) (AWRHPQFGG)
Strep-tag II (WSHPQFEK)
T7tag (MASMTGGQQMG)
TC-tag (EVHTNQDPLD)
Ty-tag (CCPGCC)
VSV-tag (YTDIEMNRLGK)
Xpress-tag (DLYDDDDK)

The expressed protein may contain a sequence acting as a solubility enhancer, for example selected from:

| | |
|---|---|
| Glutathione S-Transferase | GST |
| Small Ubiquitin-like Modifier | SUMO |
| Maltose Binding Protein | MBP |
| Fasciola hepatica 8 kDa antigen | FH8 |
| Thioredoxin | TRX |
| Solubility Enhancing Ubiquitous Tag | SNUT |
| Seventeen kilodalton protein | SKP |
| Monomeric bacteriophage T7 orc protein | MOCR |
| E coli secreted protein A | ESPA |
| N-utilization substance | NusA |
| IgG domain B0 of Protein G | GB0 |
| IgG repeat domain ZZ of Protein A | ZZ |
| Mutated dehalogenase | HaloTag |
| Phage T7 protein kinase | T7PK |
| E. coli trypsin inhibitor | Ecotin |
| Calcium-binding protein | CaBP |
| Stress-response arsenate reductase | ArsC |
| N-terminal fragment of translation initiation factor IF2 | IF2-domain 1 |
| Stress-response protein | RpoA |
| Stress-response protein | SlyD |
| Stress-response protein | Tsf |
| Stress-response protein | RpoS |
| Stress-response protein | PotD |
| Stress-response protein | Crr |
| E. coli acidic protein | msyB |
| E. coli acidic protein | yjgD |
| E. coli acidic protein | rpoD |
| T7 phage tail | P17 |
| metal-binding protein | CUSF |
| 53-amino-acid-long N-terminal extension sequence | NEXT |

The water soluble expression decoy protein may include, for example, a protein from Borrelia burgdorferi, namely outer surface protein A (OspA), which is lacking its native export signal peptide. In one embodiment, the OspA may be introduced to the N terminus of chimeric nucleic acid construct of the IMP and the amphipathic shield domain protein described herein (e.g., an EmrE-ApoAl* chimera). In one embodiment, the nucleic acid molecule may encode for a chimeric protein containing a fusion of OspA-EmrE-ApoAl. The water soluble expression decoy protein may alternatively be, but is not limited to, maltose binding protein (MBP) lacking its native export signal peptide, DnaB lacking its native export signal peptide, green fluorescent protein (GFP), and glutathione S-transferase (GST). MBP is highly soluble and larger than OspA and in one embodiment, may be positioned at the N-terminal of the chimeric nucleic acid molecule and/or protein of the present invention. The chimeric nucleic acid molecule may encode for a chimeric protein containing a fusion of MBP-EmrE-ApoAl.

The nucleic acid construct and chimeric protein of the present invention may include a flexible polypeptide linker separating the amphipathic shield domain protein, IMP, and/or water soluble expression decoy proteins and allowing for their independent folding. The linker may be approximately 15 amino acids or 60 Å in length (“4 Å per residue) but may be as long as 30 amino acids but preferably not more than 20 amino acids in length. It may be as short as 3 amino acids in length, but more preferably is at least 6 amino acids in length.

To ensure flexibility and to avoid introducing steric hindrance that may interfere with the independent folding of the fragment domain of reporter protein and the members of the putative binding pair, the linker should be comprised of small, preferably neutral residues such as Gly, Ala, and Val, but also may include polar residues that have heteroatoms such as Ser and Met, and may also contain charged residues. The first, second, and third proteins may be linked via a short polypeptide linker sequence. Suitable linkers include peptides of between about 2 and about 40 amino acids in length and may include, for example, glycine residues. Linkers may have virtually any sequence that results in a generally flexible chimeric protein.

The left flank primer and/or the right flank primer may further comprise protective elements that inhibit digestion of the left flank and/or right primers and the resulting expression construct by nucleases.

The protective elements may be selected from the following: internal phosphorothioate bonds, terminal capping groups (e.g. 5′-alkylamino, 3′-phosphate, 3′-inverted T etc.) or modified nucleotides (e.g. methylated bases, 2-aminoadenosine, base-modified bases etc.), hairpin motifs or g-quadruplexes. The protective elements may enable circularisation of the expression construct to thereby protect the expression construct from terminal nucleases. The protective elements may be buffer sequences that absorb nuclease digestion without affecting the operationally important regions of the construct such as the start and stop codons.

The left flank primer and/or the right primer may further comprise isolation elements for pulldown enrichment of the left flank and/or right primer and the resulting expression construct.

The left flank primer can be between 200 and 3000 nucleotides in length. More preferably, the left flank primer is at least 1000 nucleotides in length. Most preferably, the left flank primer is between 1000 and 3000 nucleotides in length.

The right primer can be between 100 and 3000 nucleotides in length.

The right primer may end with the B0 complementary sequence 5′-GAGAACCTGTACTTCCAGAGC-3′. Such sequences express the TEV protease cleavage site ENLYFQS.

The amplification steps may be PCR amplification or isothermal amplification, for example, loop-mediated isothermal amplification.

The two amplification steps which add A0 and B0 and then use them for amplification are separate. The two amplification steps may occur consecutively in the same reaction mixture or different reaction mixtures. Where an amplification primer is used this is generally added to the left and right flank primers to enable amplification of full length product and deplete the ratio of the flank primers, The left flank primer contains the promoter region and ribosome binding site, hance may initiate transcription and translation of proteins, but which will be truncated and not contain the sequence of the protein of interest. Thus the ration of flank primers to full length adapted constructs should be minimised to reduce the presence of short proteins.

Where the detector protein is after the POI insert (the C terminus), introduced using the right flank primers, then expression shortmers are generally not detected. The left flank primer does not contain the detection tag, and therefore remaining flanks which express short proteins sequences can not be detected.

The method disclosed may further comprise isolating the amplicon from the forward and reverse adapter primers before further amplification with the left flank and right flank primers.

The second amplification may be performed using a plurality of left flank primers and a single right flank primer to produce a population of expression constructs having a different ribosome binding sites and/or solubility tags.

Internal regions of complementarity may be used to allow a multi-part assembly. The 3′-end of one extension product and 3′-end of another extension product may hybridise to each other, allowing extension against each other. The extended ends are hence complementary, allowing further amplification of the two extension products to make a multi-part extension assembly. Rather than two primers being used to amplify one template (T1) by hybridising at each end, one primer can extend one template (T1) and the other primer a different template (T2). If the two extended ends of T1 and T2 are complementary, extension can occur to make a full length template construct which includes both templates in a contiguous sequence T1+T2 along with the primer ends. Data is shown herein using four and five part assemblies, but any number of parts can be used depending on the template length required for a particular protein and the sequence complexity of the desired strands.

Further amplification steps may be used. For example the left flank and right flank primers can be supplemented with terminal flanking primers at a higher concentration to enrich for the full length amplicons. This way, the megaprimers provide the specificity (i.e. enable a functional construct to be generated) but the inclusion of the flanking primers allows the number of moles of amplicon to be dramatically increased.

The method disclosed may further comprise combining the nucleic acid expression construct with a plurality of other expression constructs also prepared according to the method disclosed herein.

Also disclosed herein is a method of expressing a protein using a construct or population of constructs. The protein may be expressed using a cell-free system. The cell-free system may be a cell lysate. The cell-free system can be assembled from constituent components.

Also disclosed herein is a kit comprising an expression construct or population of expression constructs and components for cell-free protein expression.

Also disclosed herein is a kit comprising a population of left flank primers and a single right flank primer for amplification of a nucleic acid wherein:
i. the left flank primers each comprise a promoter sequence, a sequence encoding for a single ribosome binding site and, at its 3′ end, a sequence complementary to a nucleic acid to be amplified, wherein the population contains different ribosome binding sites; and
ii. the right flank primer comprises a terminator sequence, a sequence encoding for a stop codon and, at its 3′ end, a sequence complementary to a nucleic acid to be amplified; and
wherein the left flank and right flank primers are independently between 100 and 3000 nucleotides in length.

Following protein expression, the construct may be converted into a cloning vector. The left flank primer and/or right flank primer may contain one or more restriction sites to enable insertion into a cloning vector by ligation. Alternatively the forward adapter priming sequence and/or the reverse adapter priming sequence may contain one or more restriction sites to enable insertion into a cloning vector by ligation. Alternatively the left flank primer at the 5′ end and the right flank primer at 3′ end may contain sequences that serve as homology arms to enable insertion into a cloning vector by polymerase chain reaction.

Nucleic acid expression is the process by which information from a gene is used in the synthesis of a functional gene product that enables it to produce end products, protein or non-coding RNA. All steps in the nucleic acid expression process may be modulated (regulated), including the transcription, RNA splicing, translation, and post-translational modification of a protein.

Cell-free protein synthesis, also known as in vitro protein synthesis or CFPS, is the production of protein using biological machinery in a cell-free system, that is, without the use of living cells. CFPS environment is not constrained by a cell wall or homeostasis conditions necessary to maintain cell viability. Thus, CFPS enables direct access and control of the translation environment which is advantageous for a number of applications including co-translational solubilisation of membrane proteins, optimisation of protein production, incorporation of non-natural amino acids, selective and site-specific labelling. Due to the open nature of the system, different expression conditions such as pH, redox potentials, temperatures, and chaperones can be screened. Since there is no need to maintain cell viability, toxic proteins can be produced.

A cell-free reaction, including extract preparation, usually takes 1 to 2 days, whereas in vivo protein expression may take 1 to 2 weeks.

CFPS is an open reaction in that the lack of a cell membrane/wall allows direct manipulation of the chemical environment. Samples are easily taken, concentrations optimized, and the reaction can be monitored. There is no requirement to maintain viable cells. In contrast, once DNA is inserted into live cells, the cells need to be maintained in a viable state, and the reaction cannot be easily be assessed until it is over and the cells are lysed.

Common cell extracts are made from E. coli (ECE), rabbit reticulocytes (RRL), wheat germ (WGE), insect cells (ICE) and Yeast Kluyveromyces (the D2P system).

The production of an RNA copy from a DNA strand is called transcription, and is performed by RNA polymerases, which add one ribonucleotide at a time to a growing RNA strand as per the complementarity law of the nucleotide bases. This RNA is complementary to the template 3′→5′ DNA strand, with the exception that thymine's (T) are replaced with uracil's (U) in the RNA.

While transcription of prokaryotic protein-coding genes creates messenger RNA (mRNA) that is ready for translation into protein, transcription of eukaryotic genes leaves a primary transcript of RNA (pre-RNA), which first has to undergo a series of modifications to become a mature RNA.

In translation, messenger RNA (mRNA) is decoded in a ribosome, outside the nucleus, to produce a specific amino acid chain, or polypeptide. The mRNA carries genetic information encoded as a ribonucleotide sequence from the chromosomes to the ribosomes. The ribosome molecules translate this code to a specific sequence of amino acids. The ribosome is a multi-subunit structure containing rRNA and proteins.

The polypeptide later folds into an active protein and performs its functions in the cell. The ribosome facilitates decoding by inducing the binding of complementary tRNA anticodon sequences to mRNA codons. The tRNAs carry specific amino acids that are chained together into a polypeptide as the mRNA passes through and is read by the ribosome.

A ribosome binding site, or ribosomal binding site (RBS), is a sequence of nucleotides upstream of the start codon of an mRNA transcript that is responsible for the recruitment of a ribosome during the initiation of translation.

A terminator sequence, also known as a transcription terminator, is a section of nucleic acid sequence that marks the end of a gene or operon in genomic DNA during transcription.

Polymerase chain reaction (PCR) uses a pair of primers to direct DNA elongation toward each other at opposite ends of the sequence being amplified. These primers typically hybridise specifically to a region between 18 and 24 bases in length upstream and downstream sites of the sequence being amplified. A primer that can bind to multiple regions along the DNA will amplify without any selectivity. Primer sequences are typically chosen to uniquely select for a region of DNA by avoiding the possibility of hybridization to a similar sequence nearby.

A primer is a short single-stranded nucleic acid used in the initiation of DNA synthesis. DNA polymerase (responsible for DNA replication) enzymes are only capable of adding nucleotides to 3′-end of an existing nucleic acid, requiring a primer be bound to the template before DNA polymerase can begin a complementary strand. DNA polymerase adds nucleotides after binding to the primer and synthesises the whole complementary strand.

Electrowetting is the modification of the wetting properties of a surface (which is typically hydrophobic) with an applied electric field. Microfluidic devices for manipulating droplets or magnetic beads based on electrowetting have been extensively described. In the case of droplets in channels this can be achieved by causing the droplets, for example in the presence of an immiscible carrier fluid, to travel through a microfluidic channel defined by the walls of a cartridge or microfluidic tubing. Embedded in the walls of the cartridge or tubing are electrodes covered with a dielectric layer each of which are connected to an A/C biasing circuit capable of being switched on and off rapidly at intervals to modify the electrowetting field characteristics of the layer. This gives rise to the ability to steer the droplet along a given path.

As an alternative to microfluidic channel systems, droplets can also be generated and manipulated on planar surfaces using digital microfluidics (DMF). In contrast to channel based microfluidics, DMF utilizes alternating currents on an electrode array for moving fluid on the surface of the array. Liquids can thus be moved on an open-plan device by electrowetting. Digital microfluidics allows precise control over the droplet movements including droplet fusion and separation.

Cell-free protein synthesis, also known as in vitro protein synthesis or CFPS, is the production of peptides or proteins using biological machinery in a cell-free system, that is, without the use of living cells. The in vitro protein synthesis environment is not constrained within a cell wall or limited by conditions necessary to maintain cell viability, and enables the rapid production of any desired protein from a nucleic acid template, usually plasmid DNA or RNA from an in vitro transcription. CFPS has been known for decades, and many commercial systems are available. Cell-free protein synthesis encompasses systems based on crude lysate (Cold Spring Harb Perspect Biol. 2016 December; 8 (12): A123853) and systems based on reconstituted, purified molecular reagents, such as the PURE system for protein production (Methods Mol Biol. 2014; 1118:275-284). CFPS requires significant concentrations of biomacromolecules, including DNA, RNA, proteins, polysaccharides, molecular crowding agents, and more (Febs Letters 2013, 2, 58, 261-268).

To date, digital microfluidics, electrowetting-on-dielectric (EWoD), and electrokinesis in general have only found limited uses in cell-free biological-based applications, mostly due to biofouling, where biological components such as proteins, nucleic acids, crude cell extracts and other bioproducts adsorb and/or denature to hydrophobic surfaces. Biofouling is well known in the art to limit the ability of EWOD devices to manipulate droplets containing biomacromolecules. Wheeler and colleagues report that the maximum actuation time for droplets on EWoD devices containing biological media is 30 min before biofouling inhibits EWoD-based droplet actuation (Langmuir 2011, 27, 13, 8586-8594).

Digital microfluidics can be carried out in an air-filled system where the liquid drops are manipulated on the surface in air. However, at elevated temperatures or over prolonged periods, the volatile aqueous droplets simply dry onto the surface by evaporation. This issue is compounded by the high surface area to volume ratio of nanoliter and microliter sized drops. Hence air-filled systems are generally not suitable for protein expression where the temperature of the system needs to be maintained at a temperature suitable for enzyme activity and the duration of the synthesis needs to be prolonged for synthesized proteins levels to be detectable.

Protein expression typically requires an ample supply of oxygen. The most convenient and high yielding way to power CFPS is via oxidative phosphorylation where O₂ serves as the final electron acceptor; however, there are other ways that involve replenishing with energy molecules not involved in oxidative phosphorylation. In a confined microfluidic or digital microfluidic system of droplets, insufficient oxygen is available to enable efficient protein synthesis.

Described herein are improved methods allowing for the cell-free expression of peptides or proteins in a digital microfluidic device. Included is a method for the cell-free expression of peptides or proteins in a microfluidic device wherein the method comprises one or more droplets containing a nucleic acid template (i.e., DNA or RNA) and a cell-free system having components for protein expression in an oil-filled environment, and moving said droplets using electrokinesis. The components for the cell-free protein synthesis droplet can be pre-mixed prior to introduction to or mixed on the digital microfluidic device.

The droplet can be repeatedly moved for at least a period of 30 minutes whilst the protein is expressed. The droplet can be repeatedly moved for at least a period of two hours whilst the protein is expressed. The droplet can be repeatedly moved for at least a period of twelve hours whilst the protein is expressed. The act of moving the droplet allows oxygen to be supplied to the droplet and dispersed throughout the droplet. The act of moving improves the level of protein expression over a droplet which remains static.

The droplet can be moved using any means of electrokinesis. The droplet can be moved using electrowetting-on-dielectric (EWoD). The electrical signal on the EWoD or optical EWoD device can be delivered through segmented electrodes, active-matrix thin-film transistors, or digital micromirrors.

The filler liquid may be a hydrophobic or non-ionic liquid. For example the filler liquid may be decane or dodecane. The filler fluid may be a silicone oil such as dodecamethylpentasiloxane (DMPS). The filler liquid may contain a surfactant, for example a sorbitan ester such as Span 85.

The oil in the device can be any water immiscible liquid. The oil can be mineral oil, silicone oil, an alkyl-based solvent such as decane or dodecane, or a fluorinated oil. The oil can be oxygenated prior to or during the expression process. Alternatively, the device can be an air-filled device where droplets containing cell-free protein synthesis reagents are rapidly moved into position and fixed into an array under a humidified gas to prevent evaporation. Humidification can be achieved by enclosing or sealing the digital microfluidic device and providing on-board reagent reservoirs. Additionally, humidification can be achieved by connecting an aqueous reservoir to an enclosed or sealed digital microfluidic device. The aqueous reservoir can have a defined temperature or solute concentration in order to provide specific relative humidities (e.g., a saturated potassium sulfate solution at 30° C.).

A source of supplemental oxygen can be supplied to the droplets. For example droplets or gas bubbles containing gaseous or dissolved oxygen can be merged with the droplets during the protein expression. Additionally, a source of supplemental oxygen can be found by oxygenating the oil that is used as the filler medium. It is well-known in the art that oils such as hexadecane, HFE-7500, and others can be oxygenated to support the oxygen requirements of cell growth, especially E. coli cell growth (RSC Adv., 2017, 7, 40990-40995). Oxygenation can be achieved by aerating the oil with pure oxygen or atmospheric air.

The droplets can be formed before entering the microfluidic device and flowed into the device. Alternatively the droplets can be merged on the device. Included is a method comprising merging a first droplet containing a nucleic acid template such as a plasmid with a second droplet containing a cell-free extract having the components for protein expression to form a combined droplet capable of cell-free protein synthesis.

The droplets can be split on the device either before or after expression. Included herein is a method further comprising splitting the aqueous droplet into multiple droplets. If desired the split droplets can be screened with further additives. Included is a method wherein one or more of the split droplets are merged with additive droplets for screening.

The cell-free expression of peptides or proteins can use a cell lysate having the reagents to enable protein expression. Common components of a cell-free reaction include an energy source, a supply of amino acids, cofactors such as magnesium, and the relevant enzymes. A cell extract is obtained by lysing the cell of interest and removing the cell walls, DNA genome, and other debris by centrifugation. The remains are the cell machinery including ribosomes, aminoacyl-tRNA synthetases, translation initiation and elongation factors, nucleases, etc. Once a suitable nucleic acid template is added, the nucleic acid template can be expressed as a peptide or protein using the cell derived expression machinery. The cell lysate is supplemented with additional components, including purified enzymes.

Any particular nucleic acid template can be expressed using the system described herein. Three types of nucleic acid templates used in CFPS include plasmids, linear expression templates (LETs), and mRNA. Plasmids are circular templates, which can be produced either in cells or synthetically. LETs can be made via PCR. While LETs are easier and faster to make, plasmid yields are usually higher in CFPS. mRNA can be produced through in vitro transcription systems. The methods use a single nucleic acid template per droplet. The methods can use multiple droplets having a different nucleic acid template per droplet.

An energy source is an important part of a cell-free reaction. Usually, a separate mixture containing the needed energy source, along with a supply of amino acids, is added to the extract for the reaction. Common sources are phosphoenolpyruvate, acetyl phosphate, and creatine phosphate. The energy source can be replenished during the expression process by adding further reagents to the droplet during the process.

Thus the cell-lysate can be supplemented with additional reagents prior to the template being added. The cell-free extract having the components for protein expression would typically be produced as a bulk reagent or ‘master mix’ which can be formulated into many identical droplets prior to the distinct template being separately added to separate droplets. Common cell extracts in use today are made from E. coli (ECE), rabbit reticulocytes (RRL), wheat germ (WGE), insect cells (ICE) and Yeast Kluyveromyces (the D2P system). All of these extracts are commercially available.

Rather than originating from a cell extract, the cell-free system can be assembled from the required reagents. Systems based on reconstituted, purified molecular reagents are commercially available, for example the PURE system for protein production, and can be used as supplied. The PURE system is composed of all the enzymes that are involved in transcription and translation, as well as highly purified 70S ribosomes. The protein synthesis reaction of the PURE system lacks proteases and ribonucleases, which are often present as undesired molecules in cell extracts.

The term digital microfluidic device refers to a device having a two-dimensional array of planar microelectrodes. The term excludes any devices simply having droplets in a flow of oil in a channel. The droplets are moved over the surface by electrokinetic forces by activation of particular electrodes. Upon activation of the electrodes the dielectric layer becomes less hydrophobic, thus causing the droplet to spread onto the surface. A digital microfluidic (DMF) device set-up is known in the art, and depends on the substrates used, the electrodes, the configuration of those electrodes, the use of a dielectric material, the thickness of that dielectric material, the hydrophobic layers, and the applied voltage.

Once the CFPS reagents have been enclosed in the droplets, additional reagents can be supplied by merging the original droplet with a second droplet. The second droplet can carry any desired additional reagents, including for example oxygen or ‘power’ sources, or test reagents to which it is desired to expose to the expressed protein.

The droplets can be aqueous droplets. The droplets can contain an oil immiscible organic solvent such as for example DMSO. The droplets can be a mixture of water and solvent, providing the droplets do not dissolve into the bulk oil.

The droplets can be in a bulk oil layer. A dry gaseous environment simply dries the bubbles onto the surface during the expression process, leaving comet type smears of dried material by evaporation. Thus the device is filled with liquid for the expression process.

Alternatively, the aqueous droplets can be in a humidified gaseous environment. A device filled with air can be sealed and humidified in order to provide an environment that reduces evaporation of CFPS droplets.

The droplets containing the cell-free extract having the components for protein expression will therefore typically be in the oil filled environment before the nucleic acid templates are added to the droplets. The templates can be added by merging droplets on the microfluidic device. Alternatively, the templates can be added to the droplets outside the device and then flowed into the device for the expression process. For example the expression process can be initiated on the device by increasing the temperature. The expression system typically operates optimally at temperatures above standard room temperatures, for example at or above 29° C.

The expression process typically takes many hours. Thus the process should be left for at least 30 minutes or 1 hour, typically at least 2 hours. Expression can be left for at least 12 hours. During the process of expression the droplets should be moved within the device. The moving improves the process by mixing the reagents and ensuring sufficient oxygen is available within the droplet. The moving can be continuous, or can be repeated with intervening periods of non-movement.

Thus the aqueous droplet can be repeatedly moved for at least a period of 30 minutes or one hour whilst the protein is expressed. The aqueous droplet can be repeatedly moved for at least a period of two hours whilst the protein is expressed. The aqueous droplet can be repeatedly moved for at least a period of twelve hours whilst the protein is expressed. The act of moving the droplet allows mixing within the droplet, and allows oxygen or other reagents to be supplied to the droplet. The act of moving improves the level of protein expression over a droplet which remains static.

Digital microfluidics (DMF) refers to a two-dimensional planar surface platform for lab-on-a-chip systems that is based upon the manipulation of microdroplets. Droplets can be dispensed, moved, stored, mixed, reacted, or analyzed on a platform with a set of insulated electrodes. Digital microfluidics can be used together with analytical analysis procedures such as mass spectrometry, colorimetry, electrochemical, and electrochemiluminescense.

The droplet can be moved using any means of electrokinesis. The aqueous droplet can be moved using electrowetting-on-dielectric (EWoD). Electrowetting on a dielectric (EWoD) is a variant of the electrowetting phenomenon that is based on dielectric materials. During EWoD, a droplet of a conducting liquid is placed on a dielectric layer with insulating and hydrophobic properties. Upon activation of the electrodes the dielectric layer becomes less hydrophobic, thus causing the droplet to spread onto the surface.

The electrical signal on the EWoD or optically-activated amorphous silicon (a-Si) EWoD device can be delivered through segmented electrodes, active-matrix thin-film transistors or digital micromirrors. Optically-activated s-Si EWOD devices are well known in the art for actuating droplets (J. Adhes. Sci. Technol., 2012, 26, 1747-1771).

The oil in the device can be any water immiscible or hydrophobic liquid. The oil can be mineral oil, silicone oil, an alkyl-based solvent such as decane or dodecane, or a fluorinated oil. The air in the device can be any humidified gas.

A source of supplemental oxygen can be supplied to the droplets. For example droplets or gas bubbles containing gaseous or dissolved oxygen can be merged with the aqueous droplets during the protein expression. Alternatively the source of oxygen can be a molecular source which releases oxygen. Alternatively the droplets can be moved to an air/liquid boundary to enable increased diffusion of oxygen from a gaseous environment.

Alternatively the oil can be oxygenated. Alternatively the droplets can be presented in a humidified air filled device.

The droplet can be formed before entering the microfluidic device and flowed into the device. Alternatively the droplets can be merged on the device. Included is a method comprising merging a first droplet containing a nucleic acid template such as a plasmid with a second droplet containing a cell-free system having the components for protein expression to form the droplet.

The droplets can be split on the device either before, during or after expression. Included herein is a method further comprising splitting the droplet into multiple droplets. If desired the split droplets can be screened with further additives. Included is a method wherein one of more of the split droplets are merged with additive droplets for screening.

Through an affinity tag, such as a FLAG-tag, HIS-tag, GST-tag, MBP-tag, STREP-tag, or other form of affinity tag, CFPS-expressed proteins can be immobilized to a solid-support affinity resin and fresh batches of CFPS reagent can be delivered over the said resin. Thus, renewed reagents can be used to carry out protein synthesis, closely mimicking industrial methods of continuous flow (CF) and continuous exchange (CE) CFPS. By mimicking CF- and CE-CFPS, users can scale up their CFPS production methods.

The droplets can be actuated on a hydrophobic surface on the digital microfluidic device (ACS Nano 2018, 12, 6, 6050-6058). The hydrophobic surface can be a hydrophobic surface such as polytetrafluoroethylene (PTFE), Teflon AF (DuPont Inc), CYTOP (AGC Chemicals Inc), or FluoroPel (Cytonix LLC). The hydrophobic surface may be modified in such a way to reduce biofouling, especially biofouling resulting from exposure to CFPS reagents or nucleic acid reagents. The hydrophobic surface may also be superhydrophobic, such as NeverWet (NeverWet LLC) or Ultra-Ever Dry (Flotech Performance Systems Ltd). Superhydrophobic surfaces prevent biofouling compared with typical fluorocarbon-based hydrophobic surfaces. Superhydrophobic surfaces thus prolong the capability of digital microfluidic devices to move CFPS droplets and general solutions containing biopolymers (RSC Adv., 2017, 7, 49633-49648). The hydrophobic surface can also be a slippery liquid infused porous surface (SLIPS), which can be formed by infusing Krtox-103 oil (DuPont) with porous PTFE film (Lab Chip, 2019, 19, 2275).

Droplets can also contain additives to reduce the effects of biofouling on digital microfluidic surfaces. Specifically, droplets containing CFPS components can also contain additives such as surfactants or detergents to reduce the effects of biofouling on the hydrophobic or superhydrophobic surface of a digital microfluidic device (Langmuir 2011, 27, 13, 8586-8594). Such droplets may use antifouling additives such as TWEEN 20, Triton X-100, and/or Pluronic F127. Specifically, droplets containing CFPS components may contain TWEEN 20 at 0.1% v/v, Triton X-100 at 0.1% v/v, and/or Pluronic F127 at 0.08% w/v.

For electrowetting on dielectrics (EWoD), the change in contact angle of reagent upon the application of electric potential is an inverse function of surface tension. Thus, for low voltage EWoD operations, reduction in surface tension is achieved by addition of surfactants to reagents, which for CFPS reactions means to the lysate and to the DNA. This results in a dilution of the lysate, and it has been seen, in experiments, that diluting or otherwise adulterating the lysate results in a decrease in expression level of the protein of interest. Thus performing CFPS on DMF where the surfactants are added to the solutions being moved will necessarily result in a dilution and adulteration of the lysate and thus a decrease in the level of protein expression. In addition to being a problem in its own right, this further complicates extrapolation of on-DMF results to in-tube predictions of protein yield. An additional detriment of having to add surfactants to the samples is that this increases the time required for sample preparation, as well as increasing the potential for inconsistent results due to ‘user error,’ as there is more handling of reagents. An additional detriment of having to add surfactants to the samples is that certain downstream operations are hindered. For example, if a protein of interest is expressed in a cell-free system with a GFP₁₁ (or similar) peptide tag, it's downstream complementation with a GFP₁₋₁₀ (or similar) detector polypeptide is hindered in the presence of surfactant. Removal of the surfactant from the aqueous phase is therefore advantageous.

Rather than adding surfactants to the aqueous sample, it is instead possible to add surfactant, such as a sorbitan ester such as Span85 (e.g. Sorbitan trioleate, Sigma Aldrich, SKU 8401240025), to the oil. This has the advantages of enabling CFPS reactions to proceed on-DMF without dilution or adulteration. Additionally, it simplifies the sample preparation procedure for setting up the reactions, increasing the ease of use and the consistency of results. Using 1% w/w Span85 in dodecane allows for dilution-free CFPS reactions on-DMF, as well as dilution-free detection of the expressed non-fluorescent proteins. Other surfactants besides Span85, and oils other than dodecane could be used. A range of concentrations of Span85 could be used. Surfactants could be nonionic, anionic, cationic, amphoteric or a mixture thereof. Oils could be mineral oils or synthetic oils, including silicone oils, petroleum oils, and perfluorinated oils. Surfactants can have a detrimental effect on (1) the CFPS reactions and (2) the efficiency of the detection system (if the detection system involves complementation of a tag and detector). For example, by performing the CFPS reaction on-DMF with oil-surfactant mix, the detection of the expressed protein can also proceed without dilution and without adding aqueous surfactant. It has been shown that surfactants reduce the efficiency of some detection systems, including but not limited to the Split GFP (e.g. GFP₁₁/GFP₁₋₁₀) system, so removing surfactants from the reagent mix and instead adding them to the oil can be beneficial.

The peptide tag can be attached to the C or N terminus of the protein. The peptide tag may be one component of a green fluorescent protein (GFP). For example the peptide tag may be GFP₁₁ and the further polypeptide GFP₁₋₁₀. The peptide tag may be one component of sfCherry. The peptide tag may be sfCherry₁₁ and the further polypeptide sfCherry₁₋₁₀.

The protein may be fused to multiple tags. For example the protein may be fused to multiple GFP₁₁ peptide tags and the synthesis occurs in the presence of multiple GFP₁₋₁₀ polypeptides. For example the protein may be fused to multiple sfCherry₁₁ peptide tags and the synthesis occurs in the presence of multiple sfCherry₁₋₁₀ polypeptides. The protein of interest may be fused to one or more sfCherry₁₁ peptide tags and one or more GFP₁₁ peptide tags and the synthesis occurs in the presence of one or more GFP₁₋₁₀ polypeptides and one or more sfCherry₁₋₁₀ polypeptides.

### Devices

The manipulation of droplets by the application of electrical potential can be achieved on electrodes covered with an insulator or a dielectric or a series of insulators or dielectrics. Droplet manipulation as a result of an applied electrical potential is known as electrowetting. Electrokinesis occurs as result of a non-uniform electric field that influences the hydrostatic equilibrium of a dielectric liquid (dielectrophoresis or DEP) or a change in the contact angle of the liquid on solid surface (electrowetting-on-dielectric or EWoD). DEP can also be used to create forces on polarizable particles to induce their movement. The electrical signal can be transmitted to a discrete electrode, a transistor, an array of transistors, or a sheet of semi-conductor film whose electrical properties can be modulated by an optical signal.

EWoD phenomena occur when droplets are actuated between two parallel electrodes covered with a hydrophobic insulator or dielectric. The electric field at the electrode-electrolyte interface induces a change in the surface tension, which results in droplet motion as a result of a change in droplet contact angle. The electrowetting effect can be quantitatively treated using Young-Lippmann equation:

$$cos θ - cos {θ}_{0} = ( 1 / 2 γ LG ) c \cdot {V}^{2}$$

where θ₀ is the contact angle when the electric field across the interfacial layer is zero, γLG is the liquid-gas tension, c is the specific capacitance (given as εᵣ. ε₀/t, where εᵣ is dielectric constant of the insulator/dielectric, so is permittivity of vacuum, t is thickness) and V is the applied voltage or electrical potential. The change in contact angle (inducing droplet movement) is thus a function of surface tension, electrical potential, dielectric thickness, and dielectric constant.

When a droplet is actuated by EWOD, there are two opposing sets of forces that act upon it: an electrowetting force induced by electric field and resistant forces that include the drag forces resulting from the interaction of the droplet with filler medium and the contact line friction (ref). The minimum voltage applied to balance the electrowetting force with the sum of all drag forces (threshold voltage) is variably determined by the thickness-to-dielectric contact ratio of the insulator/dielectric, (t/εᵣ)^{1/2}. Thus, to reduce actuation voltage, it is required to reduce (t/ε)^{1/2} (i.e., increase dielectric constant or decrease insulator/dielectric thickness). To achieve low voltage actuation, thin insulator/dielectric layers must be used. However, the deposition of high quality thin insulator/dielectric layers is a technical challenge, and these thin layers are easily damaged before the desired electrowetting contact angle is large enough to drive the droplet is achieved. Most academic studies thus report the use of much higher voltages >100 V on easily fabricated, thick dielectric films (>3 μm) to effect electrowetting.

High voltage EWoD-based devices with thick dielectric films, however, have limited industrial applicability largely due to their limited droplet multiplexing capability. The use of low voltage devices including thin-film transistors (TFT) and optically-activated amorphous silicon layers (a-Si) have paved the way for the industrial adoption of EWoD-based devices due to their greater flexibility in addressing electrical signals in a highly multiplex fashion.

The driving voltage for TFTs or optically-activated α-Si are low (typically <15 V). The bottleneck for fabrication and thus adoption of low voltage devices has been the technical challenge of depositing high quality, thin film insulators/dielectrics. Hence there has been a particular need for improving the fabrication and composition of thin film insulator/dielectric devices.

Typically, the electrodes (or the array elements) used for EWOD are covered with (i) a hydrophilic insulator/dielectric and a hydrophobic coating or (ii) a hydrophobic insulator/dielectric. Commonly used hydrophobic coatings comprise of fluoropolymers such as Teflon AF 1600 or CYTOP. The thickness of this material as a hydrophobic coating on the dielectric is typically <100 nm and can have defects in the form of pinholes or a porous structure; hence, it is particularly important that the insulator/dielectric is pinhole free to avoid electrical shorting. Teflon has also been used as an insulator/dielectric, but it has higher voltage requirements due to its low dielectric constant and the thickness required to make it pinhole free. Other hydrophobic insulator/dielectric materials can include polymer-based dielectrics such as those based on siloxane, epoxy (e.g. SU-8), or parylene (e.g., parylene N, parylene C, parylene D, or parylene HT). Due to minimal contact angle hysteresis and a higher contact angle with aqueous solutions, Teflon is still used as a hydrophobic topcoat on these insulator/dielectric polymers. However, there are difficulties in reliably producing <1 micron pinhole-free coatings of parylene or SU-8; thus, the thickness of these materials is typically kept at a 2-5 microns at the cost of increased voltage requirements for electrowetting. It has also been reported that traditional EWoD devices with parylene C are easily broken and unstable for repeated droplet manipulation with cell culture medium. Multi-layer insulator devices deposited with metal-oxide and parylene C films have been used to produce a more robust insulator/dielectric and enable operations with lower applied voltages. Inorganic materials, such metal oxides and semiconductor oxides, commonly used in the CMOS industry as “gate dielectrics”, have been used as insulator/dielectric for EWoD devices. They offer the advantage of utilizing standard cleanroom processes for thin film depositions (<100 nm). These materials are inherently hydrophilic, requiring an additional hydrophobic coating, and can be prone to pinhole formation as a result of thin film layer deposition process. Together with the need for lower voltage operations of EWoD, recent developmental work has focused on (1) using materials with improved dielectric properties (e.g., using high-dielectric constant insulators/dielectrics), (2) optimizing the fabrication process to make the insulator/dielectric pinhole free to avoid dielectric breakdown.

Operation of EWoD devices suffers from contact angle saturation and hysteresis, which is believed to be brought about by either one or combination of these phenomena: (1) entrapment of charges in the hydrophobic film or insulator/dielectric interface, (2) adsorption of ions, (3) thermodynamic contact angle instabilities, (4) dielectric breakdown of dielectric layer, (5) the electrode-electrode-insulator interface capacitance (arising from the double layer effect), and (6) fouling of the surface (such as by biomacromolecules). One of the adverse effects of this hysteresis is reduced operational lifetime of the EWoD-based device.

Contact angle hysteresis is believed to be a result of charge accumulation at the interface or within the hydrophobic insulator after several operations. The required actuation voltage increases due to this charging phenomenon resulting in eventual catastrophic dielectric breakdown. The most probable explanation is that pinholes at the insulator/dielectric may allow the liquid to come into contact with the electrode causing electrolysis. Electrolysis is further facilitated by pinhole-prone or porous hydrophobic insulators.

Most of the studies to understand contact angle hysteresis on EWoD have been conducted on short time scales and with low conductivity solutions. Long duration actuations (e.g., >1 hour) and high conductivity solutions (e.g., 1 M NaCl) could produce several effects other than electrolysis. The ions in solution can permeate through the hydrophobic coat (under the applied electric field) and interact with the underlying insulator/dielectric. Ion permeation can result in (1) change in dielectric constant due to charge entrapment (which is different from interfacial charging) and (2) change in surface potential of a pH sensitive metal oxide. Both can result in reduction of electrowetting forces to manipulate aqueous droplets, leading to contact angle hysteresis. The inventors have previously found that the damage from high conductivity solutions reduces or disables electrowetting on electrodes by inhibiting the modulation of contact angle when an electric field is applied.

An electrokinetic device includes a first substrate having a matrix of electrodes, wherein each of the matrix electrodes is coupled to a thin film transistor, and wherein the matrix electrodes are overcoated with a functional coating comprising: a dielectric layer in contact with the matrix electrodes, a conformal layer in contact with the dielectric layer, and a hydrophobic layer in contact with the conformal layer; a second substrate comprising a top electrode; a spacer disposed between the first substrate and the second substrate and defining an electrokinetic workspace; and a voltage source operatively coupled to the matrix electrodes.

The dielectric layer may comprise silicon dioxide, silicon oxynitride, silicon nitride, hafnium oxide, yttrium oxide, lanthanum oxide, titanium dioxide, aluminum oxide, tantalum oxide, hafnium silicate, zirconium oxide, zirconium silicate, barium titanate, lead zirconate titanate, strontium titanate, or barium strontium titanate. The dielectric layer may be between 10 nm and 100 μm thick. Combinations of more than one material may be used, and the dielectric layer may comprise more than one sublayer that may be of different materials.

The conformal layer may comprise a parylene, a siloxane, or an epoxy. It may be a thin protective parylene coating in between the insulating dielectric and the hydrophobic coating. Typically, parylene is used as a dielectric layer on simple devices. In this invention, the rationale for deposition of parylene is not to improve insulation/dielectric properties such as reduction in pinholes, but rather to act as a conformal layer between the dielectric and hydrophobic layers. The inventors find that parylene, as opposed to other similar insulating coatings of the same thickness such as PDMS (polydimethylsiloxane), prevent contact angle hysteresis caused by high conductivity solutions or solutions deviating from neutral pH for extended hours. The conformal layer may be between 10 nm and 100 μm thick.

The hydrophobic layer may comprise a fluoropolymer coating, fluorinated silane coating, manganese oxide polystyrene nanocomposite, zinc oxide polystyrene nanocomposite, precipitated calcium carbonate, carbon nanotube structure, silica nanocoating, or slippery liquid-infused porous coating.

The elements may comprise one or more of a plurality of array elements, each element containing an element circuit; discrete electrodes; a thin film semiconductor in which the electrical properties can be modulated by incident light; and a thin film photoconductor whose properties can be modulated by incident light.

The functional coating may include a dielectric layer comprising silicon nitride, a conformal layer comprising parylene, and a hydrophobic layer comprising an amorphous fluoropolymer. This has been found to be a particularly advantageous combination.

The electrokinetic device may include a controller to regulate a voltage provided to the individual matrix electrodes. The electrokinetic device may include a plurality of scan lines and a plurality of gate lines, wherein each of the thin film transistors is coupled to a scan line and a gate line, and the plurality of gate lines are operatively connected to the controller. This allows all the individual elements to be individually controlled.

The second substrate may also comprise a second hydrophobic layer disposed on the second electrode. The first and second substrates may be disposed so that the hydrophobic layer and the second hydrophobic layer face each other, thereby defining the electrokinetic workspace between the hydrophobic layers.

The method is particularly suitable for aqueous droplets with a volume of 1 μL or smaller.

The EWoD-based devices shown and described below are active matrix thin film transistor devices containing a thin film dielectric coating with a Teflon hydrophobic top coat. These devices are based on devices described in the E Ink Corp patent filing on “Digital microfluidic devices including dual substrate with thin-film transistors and capacitive sensing”, US patent application no 2019/0111433, incorporated herein by reference. Described herein are electrokinetic devices, including:
a first substrate having a matrix of electrodes, wherein each of the matrix electrodes is coupled to a thin film transistor, and wherein the matrix electrodes are overcoated with a functional coating comprising:
a dielectric layer in contact with the matrix electrodes,
a conformal layer in contact with the dielectric layer, and
a hydrophobic layer in contact with the conformal layer;
   a second substrate comprising a top electrode;
   a spacer disposed between the first substrate and the second substrate and defining an electrokinetic workspace; and
   a voltage source operatively coupled to the matrix electrodes;

Described herein is an electrokinetic device, including:
a first substrate having a matrix of electrodes, wherein each of the matrix electrodes is coupled to a thin film transistor, and wherein the matrix electrodes are overcoated with a functional coating comprising:
one or more dielectric layer(s) comprising silicon nitride, hafnium oxide or aluminum oxide in contact with the matrix electrodes,
a conformal layer comprising parylene in contact with the dielectric layer, and
a hydrophobic layer in contact with the conformal layer;
   a second substrate comprising a top electrode;
   a spacer disposed between the first substrate and the second substrate and defining an electrokinetic workspace; and
   a voltage source operatively coupled to the matrix electrodes;

The electrokinetic devices as described may be used with other elements, such as for example devices for heating and cooling the device or reagent cartridges for the introduction of reagents as needed.

Example Protein Expression and purification process outline

1. User designs a DNA construct
1.1. Choose a gene of interest
1.2. Choose flanking elements
   1.2.1. Detection tag (N-terminal, C-terminal, internal) [required]
   1.2.2. Purification tags (His, Strep, other) [optional]
   1.2.3. Solubility tags (SUMO, MBP, GST, TRX, other) [optional]
   1.3. Prepare gene sequence as described herein.
      2. User loads eDrop cartridge
2.1. Input DNA construct(s)
2.2. Input CFPS reagent(s)
2.3. Input paramagnetic beads (streptactin or Ni-NTA coated)
2.4. Input other required reagents
   3. eDrop combines DNA construct(s) and CFPS reagent(s) and protein expression occurs in droplets on the EWOD device.
3.1. 4-6 hours
   4. Droplets now containing expressed protein are contacted with droplets containing paramagnetic beads coated with the appropriate moiety
4.1. Strep tag with streptavidin, neutravidin, or streptactin coated beads
   4.1.1. Preferably streptactin coated beads
4.2. His tag with Ni-NTA coated beads
   5. Purification occurs
5.1. Magnetic stage engages and pellets magnetic beads
5.2. Supernatant is removed
5.3. Wash droplet contacted with magnetic bead pellet
5.4. Magnetic stage disengages and the droplet is moved to resuspend and wash magnetic beads
5.5. Steps 5.1 to 5.4 repeated
5.6. Magnetic stage engages to pellet magnetic beads, supernatant removed and elution droplet contacted with bead pellet
5.7. Magnetic stage engages and the eluted protein is moved to a harvest port in a droplet

Each droplet on the device contains a population of nucleic acid expression constructs having the expression sequence of choice and a variety of RBS sites. The CFPS reagent droplets can contain a variety of cell lysates or purified components. A subset of the CFPS reagents should allow expression using one or more of the available nucleic acid templates. Most of the templates will not be expressed in each of the droplets, and many of the droplets will not be expressed. However a subset of the droplets will enable expression, and the droplets allowing expression can be identified and the protein harvested.

Disclosed herein is therefore a method for protein expression on an array of electrodes.

### EXAMPLES

### Step 1: AdaptPCR

PCR reaction designed to add a universal pair of flanking adapters to a region of interest (e.g. protein coding sequence, exon, ORF etc). The template can be amplified from a DNA sample, such as genomic DNA or a cDNA library, or can be a synthetic sample such as an assembled strand or a pool of oligonucleotides. In principle, the adapted region can be any length, but for practical purposes, the typical range would be 1000-5000 bp. As DNA manufacture techniques, for example phosphoramidite DNA synthesis or enzymatic DNA synthesis, improve then the typical adapted range may expand upwards due to wider availability of longer templates.

Add flanking adapters TEV and C3. Although this is an arbitrary choice, it does confer two main advantages, i) the adaptPCR is robust with few artefacts, and ii) the inclusion of TEV and C3 in the final expression cassette allows the digestion of the target protein to remove exogenous peptide regions used as detection and purification tags utilised during the CFPS expression that may otherwise inhibit the function of certain proteins.

The adaptPCR primers have a loci-specific head and universal TEV or C3 tail. These primers are short and can be synthesised easily (by chemical or enzymatic means). The loci specific head portion of the primers vary in length between 17-39 nucleotides and the TEV and C3 sequences add 21 nucleotides to the tail of the primers. Thus, the overall length is in the region of 38-60 nucleotides.

The flanking regions of the adaptPCR amplicon allows targeting in the next step by megaprimers. This way, any POI can be made compatible with a library of flank primers that can generate constructs which code for many fusion variants of that protein of interest.

No purification is required, the adaptPCR reaction is used directly in the next step.

The primer sequences can include sequences:

5′-GAGAACCTGTACTTCCAGAGC-3′
5′-TCCTTGGAACAGAACCTCGAG

### Step 2: Megaprimer PCR

A pair of megaprimers are added to the adaptPCR amplicon and subjected to further cycles of PCR. Each of the megaprimers are (100-3000 nt) DNA molecules that have either TEV or C3 at their 3′ termini and also encode for the regulatory elements required to support cell-free transcription/translation.

The megaprimer TEV and C3 ends are complementary to the adaptPCR amplicon which when extended in the presence of the adaptPCR template results in the formation of the full-length UMA-LEC expression construct.

The full-length expression construct comprises the POI flanked on 5′ side by a megaprimer encoding the transcription start and ribosome binding sites, and on 3′ side by a megaprimer encoding the transcription stop and terminator sites. A variety of other elements can be encoded into either 5′ or 3′ flanking arm of the expression construct, depending on requirements and also depending on compatibility of the expression construct with the target lysate in which transcription/translation is anticipated to be conducted in.

A shortlist (not exhaustive) of the type of elements commonly encoded in the megaprimers is given below:
Detection tags (e.g. sfGFP, GFP₁₁, LBT, HiBit).
Purification tags (e.g. HisTag, StrepTag).
Linkers and spacers (e.g. short polypeptide regions that space regulatory elements apart).
Solubility tags (e.g. peptides expressing with fusion proteins that improve aqueous solubility and folding).
Alternative ribosome binding sites (RBS) and internal ribosome entry sites (IRES) that tailor UMA-LEC expression of the same POI in different lysates.

Step 1 and step 2 of the process can be conducted in a ‘two-step single-pot’ format, or a ‘two-step two-pot’ format, depending on whether intermediate purification is required, and the level of impurities that can be tolerated in the sample by the CFPS expression system. The ‘two-step two pot’ version requires the adaptPCR and megaprimer-PCR reactions to be run independently of each other, and has a requirement for an intermediate cleanup. For these reasons, this method generates less artefacts (e.g. >90% correct product) and UMA-LECs are delivered at higher final concentration. The ‘two-step one-pot’ version involves the spiking of megaprimers into the adaptPCR reaction and continuing the thermocycling in the same vessel. As a result, this method is quicker but typically results in lower yield and a slightly less pure final construct (e.g. >80% correct product).

The double stranded template having the gene of interest can be synthesized having protease cleavage sites at 5′- and 3′-ends. The protease cleavage sites can be for example 3C and TEV. The template can be made using amplification or can be synthesized.

Also described herein is a kit comprising a first double stranded nucleic acid adapter having a sequence coding for a first protease cleavage site at one end of the nucleic acid and a second double stranded nucleic acid adapter having a sequence coding for a second protease cleavage site at one end of the nucleic acid. These first and second nucleic acid adapters can act as primers for a template having protease cleavage sequences at 5′- and 3′-ends. Amplification gives an amplicon having the first and second nucleic acid adapters flanking the double stranded templates. The first and second adapters can be independently between 100 and 3000 nucleotides in length.

The composition can also contain further primers enabling selective amplification of the contiguous template and first and second adapters.

As both the adaptPCR and UMA-PCR steps generate long amplicons, they are amenable to
either thermocycling PCR or isothermal amplification methodologies. Versions of this approach could be imagined that deliver the final UMA-LEC in a circular form, thereby making it a nuclease resistant expression template.

The method is amenable to functionalizing the terminal ends of the megaprimers to make them nuclease resistant, or to allow pulldown enrichment (e.g. internal phosphorothioate bonds or biotin modification respectively).

Megaprimers are manufactured themselves by PCR and as such their construction is extremely flexible in terms of the type of payload (e.g. number of regulatory elements), length of each of the flanking arms, GC content and repetitiveness etc. The megaprimer arms can be made by targeting up- and down-stream regions of common cloning vectors but are also amenable to complete de novo design and in vitro synthesis.

Specific embodiments may include the coding sequences for example:

| | outline DNA | Left Megaprimer Coding Sequence | Right Megaprimer Codon Sequence |
|---|---|---|---|
| N-SOL | Construct | (5′ to 3′) | (5′ to 3′) |
| P17 | P17-//-DET- | ATGTCAAAGGAAAAAAGAAAGAACGAG | /GAGAACCTGTACTTCCAGAGCGGTGGTG |
| | STREP | AGCAGCACAAATGCGACAAATACCAAG | GAGGGAGCGGTGGGGGAGGCTCTGGGG |
| | | CAGTGGCGCGACGAGACCAAGGGTTTC | GAGGAGGAAGCGGTGAAACCATCCAGTT |
| | | CGCGACGAGGCAAAACGTTTCAAAAACA | ACAAGAACACGCCGTGGCCAAATATTTC |
| | | CTGCGGGAGGAGGCGGCTCAGAAGGCG | ACCGAAGAAGCGGCTGCCAAGGAGGCG |
| | | GAGGATCTGAGGGCGGTGGGTCAGAGC | GCCGCAAAAGAGGCGGCTGCAAAATGG |
| | | TCGAGGTTCTOTTCCAAGGACCT/ | AGTCATCCTCAGTTCGAAAAATAA |
| CUSF | CUSF-//-DET- | ATGTCAAAGGAAAAAAGAGCTAACGAAC | /GAGAACCTGTACTTCCAGAGCGGTGGTG |
| | STREP | ATCATCATGAAACCATGAGCGAAGCACA | GAGGGAGCGGTGGGGGAGGCTCTGGGG |
| | | ACCACAGGTTATTAGCGCCACTGGCGTG | GAGGAGGAAGCGGTGAAACCATCCAGTT |
| | | GTAAAGGGTATCGATCTGGAAAGCAAAA | ACAAGAACACGCCGTGGCCAAATATTTC |
| | | AAATCACCATCCATCACGATCCGATTGCT | ACCGAAGAAGCGGCTGCCAAGGAGGCG |
| | | GCCGTGAACTGGCCGGAGATGACCATG | GCCGCAAAAGAGGCGGCTGCAAAATGG |
| | | CGCTTTACCATCACCCCGCAGACGAAAA | AGTCATCCTCAGTTCGAAAAATAA |
| | | TGAGTGAAATTAAAACCGGCGACAAAGT | |
| | | GGCGTTTAATTTTGTCCAGCAGGGCAAC | |
| | | CTTTCTTTATTACAGGATATTAAAGTCAGC | |
| | | CAGGGAGGCGGCTCAGAAGGCGGAGGA | |
| | | TCTGAGGGCGGTGGGTCAGAGCTCGAG | |
| | | GTTCTOTTCCAACCACCT/ | |
| FH8 | FH8-//-DET- | ATGTCAAAGGAAAAAAGACCCAGTGTAC | /GAGAACCTGTACTTCCAGAGCGGTGGTG |
| | STREP | AAGAAGTAGAAAAACTATTACATGTTCTA | GAGGGAGCGGTGGGGGAGGCTCTGGGG |
| | | GATAGGAATGGAGACGGCAAGGTGTCT | GAGGAGGAAGCGGTGAAACCATCCAGTT |
| | | GCCGAAGAATTAAAAGCATTTGCTGACG | ACAAGAACACGCCGTGGCCAAATATTTC |
| | | ATTCCAAATGTCCTITGGACTCAAATAAA | ACCGAAGAAGCGGCTGCCAAGGAGGCG |
| | | ATTAAAGCTITTATAAAAGAACATGATAA | GCCGCAAAAGAGGCGGCTGCAAAATGG |
| | | AAATAAGGATGGTAAACTTGATTTAAAAG | AGTCATCCTCAGTTCGAAAAATAA |
| | | AGCTTGTAAGTATTTTGTCATCTGGAGGC | |
| | | GGCTCAGAAGGCGGAGGATCTGAGGGC | |
| | | GGTGGGTCAGAGCTCGAGOTTCTOTTCC | |
| | | AAGGACCT/ | |
| TRX | TRX-//-DET- | ATGTCAAAGGAAAAAAGATCAGATAAAA | /GAGAACCTGTACTTCCAGAGCGGTGGTG |
| | STREP | TAATTCATITAACAGATGATAGTTTTGATA | GAGGGAGCGGTGGGGGAGGCTCTGGGG |
| | | CTGATGTATTGAAAGCAGATGGAGCTAT | GAGGAGGAAGCGGTGAAACCATCCAGTT |
| | | CCTCGTTGATTTTTGGGCTGAATGGTGTG | ACAAGAACACGCCGTGGCCAAATATTTC |
| | | GACCCTGTAAAATGATTGCACCTATTTTA | ACCGAAGAAGCGGCTGCCAAGGAGGCG |
| | | GATGAAATTGCTGATGAATATCAAGGTA | GCCGCAAAAGAGGCGGCTGCAAAATGG |
| | | AATTAACAGTCGCTAAATTAAATATTGAT | AGTCATCCTCAGTTCGAAAAATAA |
| | | CAAAATCCAGGTACTGCTCCAAAATATG | |
| | | GAATTAGAGGAATACCTACTCTTTTATTAT | |
| | | TTAAAAATGGCGAAGTGGCTGCAACAAA | |
| | | AGTGGGAGCTTTATCTAAAGGTCAACTA | |
| | | AAAGAATTITTAGATGCAAATCTTGCAGG | |
| | | AGGCGGCTCAGAAGGCGGAGGATCTGA | |
| | | GGGCGGTGGGTCAGAGCTCGAGGTTCTO | |
| | | TTCCAAGGACCT/ | |
| ZZ | ZZ-//-DET- | ATGTCAAAGGAAAAAAGAGTTGATAACA | /GAGAACCTGTACTTCCAGAGCGGTGGTG |
| | STREP | AATTCAATAAAGAACAGCAAAACGCATA | GAGGGAGCGGTGGGGGAGGCTCTGGGG |
| | | TTACGAGATTCTTCATCTGCCGAATTTGA | GAGGAGGAAGCGGTGAAACCATCCAGTT |
| | | ATGAAGGCCAACGTAATGCGTTTATCCA | ACAAGAACACGCCGTGGCCAAATATTTC |
| | | GTCCCTTAAAGACGATCCTTCCCAGTCTG | ACCGAAGAAGCGGCTGCCAAGGAGGCG |
| | | CGAACTTGTTAGCGGAGGCCAAAAAATT | GCCGCAAAAGAGGCGGCTGCAAAATGG |
| | | AAACGATGCCCAAGCTCCCAAGGTGGAT | AGTCATCCTCAGTTCGAAAAATAA |
| | | AATAAGTTCAATAAGGAACAACAGAATG | |
| | | CTTTTTACGAAATCTTGCACCTGCCCAAT | |
| | | CTTAACGAAGAACAACGCAATGCTTTCA | |
| | | TTCAAAGTCTGAAAGACGATCCCTCGCA | |
| | | AAGTGCGAACTTATTGGCCGAGGCTGAG | |
| | | AAACTTAATGACGCTCAAGCGCCCAAGG | |
| | | GAGGCGGCTCAGAAGGCGGAGGATCTG | |
| | | AGGGCGGTGGGTCAGAGCTCGAGOTTCT | |
| | | CTTCCAAGGACCT/ | |
| HSUMO3 | SUMO-//-DET- | ATGTCAAAGGAAAAAAGAAGCGAAGAA | /GAGAACCTGTACTTCCAGAGCGGTGGTG |
| | STREP | AAACCCAAGGAAGGCGTCAAAACCGAA | GAGGGAGCGGTGGGGGAGGCTCTGGGG |
| | | AACGATCATATCAATTTAAAGGTTGCCGG | GAGGAGGAAGCGGTGAAACCATCCAGTT |
| | | GCAAGATGGCAGCGTAGTCCAGTTCAAG | ACAAGAACACGCCGTGGCCAAATATTTC |
| | | ATTAAGCGTCACACGCCGTTGAGTAAAC | ACCGAAGAAGCGGCTGCCAAGGAGGCG |
| | | TGATGAAAGCCTATTGCGAGCGTCAGGG | GCCGCAAAAGAGGCGGCTGCAAAATGG |
| | | GCTTAGTATGCGCCAAATTCGCTTCCGCT | AGTCATCCTCAGTTCGAAAAATAA |
| | | TCGACGGACAGCCAATCAATGAAACGG | |
| | | ATACTCCTGCTCAACTGGAAATGGAAGA | |
| | | TGAGGACACCATTGATGTGTTTCAGCAA | |
| | | CAAACGGGAGGCGTTCCAGAGTCTTCAC | |
| | | TTGCAGGACACAGCTTCGGAGGCGGCTC | |
| | | AGAAGGCGGAGGATCTGAGGGCGGTGG | |
| | | GTCAGAGCTCCAGGTTCTOTTCCAAGGA | |
| | | CCT/ | |
| SNUT | SNUT-//-DET- | ATGTCAAAGGAAAAAAGAAAGCCTCACA | /GAGAACCTGTACTTCCAGAGCGGTGGTG |
| | STREP | TTGACAATTATTTACACGACAAGGACAAA | GAGGGAGCGGTGGGGGAGGCTCTGGGG |
| | | GACGAGCGCATTGAGCAATACGACAAA | GAGGAGGAAGCGGTGAAACCATCCAGTT |
| | | AATGTCAAGGAACAAGCAAGCAAGGAC | ACAAGAACACGCCGTGGCCAAATATTTC |
| | | AAGAAACAGCAGGCGAAGCCTCAAATC | ACCGAAGAAGCGGCTGCCAAGGAGGCG |
| | | CCCAAGGACAAAAGTAAAGTCGCTGGGT | GCCGCAAAAGAGGCGGCTGCAAAATGG |
| | | ACATCGAGATCCCGGATGCAGACATTAA | AGTCATCCTCAGTTCGAAAAATAA |
| | | GGAGCCCGTCTATCCTGGACCTGCAACA | |
| | | CCTGAGCAGCTTAATCGCGGGGTGTCGT | |
| | | TTGCGGAAGAAAATGAGTCGTTGGACGA | |
| | | CCAGAATATCAGCATCGCAGGCCACACC | |
| | | TTTATCGACCGCCCCAATTATCAGTTCAC | |
| | | AAACTTGAAAGCGGCCAAGAAGGGTTCA | |
| | | ATGGTATATTTCAAGGTTGGAAATGAGAC | |
| | | GCGCAAATACAAGATGACAAGTATCCGT | |
| | | GACGTCAAACCTACTGACGTAGAAGTAC | |
| | | TTGATGGTTCCGGAGGCGGCTCAGAAGG | |
| | | CGGAGGATCTGAGGGCGGTGGGTCAGA | |
| | | GCTCGAGGTTCTOTTCCAAGOACCT/ | |
| No SOL | //-DET-STREP | ATGTCAAAGGAAAAAAGACTCGAGOTTC | /GAGAACCTGTACTTCCAGAGCGGTGGTG |
| | | TGTTCCAAGGACCT/ | GAGGGAGCGGTGGGGGAGGCTCTGGGG |
| | | | GAGGAGGAAGCGGTGAAACCATCCAGTT |
| | | | ACAAGAACACGCCGTGGCCAAATATTTC |
| | | | ACCGAAGAAGCGGCTGCCAAGGAGGCG |
| | | | GCCGCAAAAGAGGCGGCTGCAAAATGG |
| | | | AGTCATCCTCAGTTCGAAAAATAA |

Constructs may be codon optimized for expression in particular conditions. Tag sequences may be codon optimized. For example the strep sequence WSHPQFEK may be coded for by the sequence TGGAGTCATCCTCAGTTCGAAAAA.

The right flank adapter may include the elements of a protease cleavage site, a spacer, a detection tags (for example ccGFP₁₁), a spacer and purification tag (for example strep or strep II)

The amino acid sequence coded by the right flank
adapter may be
ENLYFQSGGGGSGGGGSGGGGSGETIQLQEHAVAKYFTEEAAAKEAAAKE
AAAKWSHPQFEK.

Constructs may be used having a low GC % sequence after the expression start. The protein of interest may be appended with a sequence such as TCAAAGGAAAAAAGA (SKEKR) which aids expression. sequence may have for example less than 35% GC over a string of at least 15 nucleotides. The expression start sequence may be ATGTCAAAGGAAAAAAGA

Specific optimization has identified 28 PCR cycles as the optimum number to give sufficient template amplification, but without an increase in shorter by-products that give expression shortmers. The number of cycles may be between 25-28 cycles. Fewer cycles gives insufficient material for subsequent expression, more cycles gives an increase in shortened extension products.

Specific optimization has identified the following ratios and concentrations of templates, flanking primers and amplification primers:
Flank primers Initial conc=20 nM Vol used=1 μL MP final con in 60 μL=0.3 nM
Terminal Primer Initial conc=1000 μM Vol used=0.018 μL Terminal Primer final conc=300 nM
Template intial conc=2 nM Vol used=1 μL Temp final Conc in 60 μL=0.03 nM
Ratio: Template=1 Flanks=10 Terminal Primer=10,000

Thus the amplification primers can be used in excess compared to the flanking primers. For example at least 100 fold excess in concentration or at least 1000 fold excess of the amplification primers can be used in order to convert the flanking primers into full length amplicons and lower the presence of truncated transcripts.

### Example 1: Using adaptPCR to Prepare 48×UMA-LEC Expression Constructs

### Materials:

### DNA Templates:

| | Template sequence | Sequence |
|---|---|---|
| Protein name | (5′ to 3′) ID number | length |
| Human alpha -Galactosidase A_sfGFP | 3 | 2361 |
| Human csbBroadEn_08244 CRBN_sfGFP | 4 | 2463 |
| Human Cystatin C_sfGFP | 5 | 1587 |
| Human Glucosylceramidase/GBA_sfGFP | 6 | 2760 |
| Human Heparanase/HPSE_sfGFP | 7 | 2778 |
| Human Iduronate 2-Sulfatase_sfGFP | 8 | 2799 |
| Human matrix metalloproteinase-1 (MMP-1)_sfGFP | 9 | 2358 |
| Human TFPI_sfGFP | 10 | 1902 |
| Human VHL_sfGFP | 11 | 1665 |
| Human Cathepsin A/Lysosom Carboxypeptidase | 12 | 2142 |
| A_sfGFP | | |
| TEV P1 protease_sfGFP | 13 | 2061 |
| B. thermoproteolyticus Thermolysin_sfGFP | 14 | 2793 |
| Human Trypsin 1/PRSS1 (serine protease 1)_sfGFP | 15 | 1890 |
| Arthrobacter sp. alcohol dehydrogenase_sfGFP | 16 | 1659 |
| Bacillus subtilis homoserine dehydrogenase | 17 | 2361 |
| (hom)_sfGFP | | |
| Colletotrichum aenigma Diacetyl reductase_sfGFP | 18 | 1983 |
| Drosophila melanogaster Glycerol-3-phosphate | 19 | 2199 |
| dehydrogenase 1_sfGFP | | |
| Rasamsonia emersonii Propanediol-phosphate | 20 | 1920 |
| dehydrogenase_sfGFP | | |
| Mouse lactate dehydrogenase A (LDH-A)_sfGFP | 21 | 1545 |
| Arabidopsis thaliana malate dehydrogenase | 22 | 2358 |
| (MDH)_sfGFP | | |
| Arabidopsis thaliana isocitrate dehydrogenase | 23 | 2397 |
| (ICDH)_sfGFP | | |
| Cucumis melo HMG-CoA reductase_sfGFP | 24 | 2910 |
| Aspergillus niger glucose oxidase_sfGFP | 25 | 2961 |
| Zea mays L-gulonolactone oxidase 2_sfGFP | 26 | 2943 |
| Arthrobacter sp. xanthine oxidase_sfGFP | 27 | 2688 |
| Colletotrichum gloeosporioides glyceraldehyde-3- | 28 | 2163 |
| phosphate dehydrogenase_sfGFP | | |
| Xenopus tropicalis biliverdin reductase B (blvrb)_sfGFP | 29 | 1707 |
| Drosophila innubila protoporphyrinogen oxidase_sfGFP | 30 | 2580 |
| Salmo gairdneri monoamine oxidase (MAO)_sfGFP | 31 | 2646 |
| Escherichia coli dihydrofolate reductase (folA)_sfGFP | 32 | 1626 |
| Mesocricetus auratus pipecolic acid and sarcosine | 33 | 1920 |
| oxidase (Pipox)_sfGFP | | |
| Sparassis crispa Pyrimidodiazepine synthase_sfGFP | 34 | 1863 |
| Sturnus vulgaris electron-transferring-flavoprotein | 35 | 3006 |
| dehydrogenase (ETFDH)_sfGFP | | |
| Human methyl-CpG binding domain protein 2 | 36 | 2055 |
| (MBD2)_sfGFP | | |
| Momordica charantia cytokinin dehydrogenase 4_sfGFP | 37 | 2721 |
| Glycine max proline dehydrogenase (PDH)_sfGFP | 38 | 2643 |
| Glycine max chalcone reductase CHR1 (CHR1)_sfGFP | 39 | 2094 |
| Schizosaccharomyces cryophilus OY26 NADPH- | 40 | 1569 |
| hemoprotein reductase_sfGFP | | |
| Oryza sativa Japonica Group ubiquinol oxidase 4_sfGFP | 41 | 2157 |
| Human renalase, FAD dependent amine oxidase | 42 | 2094 |
| (RNLS)_sfGFP | | |
| Rattus norvegicus catechol-O-methyltransferase | 43 | 1941 |
| (Comt)_sfGFP | | |
| Mus musculus aminolevulinic acid synthase 2_sfGFP | 44 | 2865 |
| Arabidopsis thaliana Hypoxanthine-guanine | 45 | 1743 |
| phosphoribosyltransferase (HGPT)_sfGFP | | |
| Bovine TdT_sfGFP | 46 | 2676 |
| Bovine TdT_del_BRCT_sfGFP | 47 | 2235 |
| Fish TdT_sfGFP | 48 | 2634 |
| Fish TdT_del_BRCT_sfGFP | 49 | 2220 |
| Saccharomyces cerevisiae inorganic diphosphatase | 50 | 2010 |
| IPP1(YIPP)_sfGFP | | |

### AdaptPCR Primer Mixes:

| PCR_F/ | | |
|---|---|---|
| R_mix | PCR_F sequence (5′ to 3′) | PCR_R sequence (5′ to 3′) |
| M0001 | GAGAACCTGTACTTCCAGAGCCCTG | TCCTTGGAACAGAACCTCGAGAA |
| | GAGCACGTGCTCTG | GCAAGTCTTTCAGCGACATTTG |
| M0002 | GAGAACCTGTACTTCCAGAGCGCCG | TCCTTGGAACAGAACCTCGAGTTT |
| | GAGAAGGCGATCAG | GTCGGGGGAAATTTCATCTTC |
| M0003 | GAGAACCTGTACTTCCAGAGCGCCG | TCCTTGGAACAGAACCTCGAGGG |
| | GGCCATTGCGTGC | CATCCTGGCACGTCGAC |
| M0004 | GAGAACCTGTACTTCCAGAGCGAGTT | TCCTTGGAACAGAACCTCGAGCT |
| | TAGCTCGCCCAGCC | GGCGACGCCAAAGATATG |
| M0005 | GAGAACCTGTACTTCCAGAGCCTGCT | TCCTTGGAACAGAACCTCGAGGA |
| | GCGCAGCAAACC | TACACGCCGCTACTTTGGC |
| M0006 | GAGAACCTGTACTTCCAGAGCCCGC | TCCTTGGAACAGAACCTCGAGAG |
| | CACCGCGTACTGG | GCATCAGCAGTTGAAACAAGTC |
| M0007 | GAGAACCTGTACTTCCAGAGCCAGG | TCCTTGGAACAGAACCTCGAGAT |
| | AATTTTTTGGCTTGAAAGTG | TCTTACGACAGTTGAACCATGAG |
| M0008 | GAGAACCTGTACTTCCAGAGCATTTA | TCCTTGGAACAGAACCTCGAGAC |
| | CACAATGAAAAAAGTACACGCC | ATAAACAAGAGATGGAATCCAGG |
| M0009 | GAGAACCTGTACTTCCAGAGCCCCC | TCCTTGGAACAGAACCTCGAGAT |
| | GCCGCGCAGAGAA | CTCCCATGCGTTGGTGGGC |
| M0010 | GAGAACCTGTACTTCCAGAGCAAACG | TCCTTGGAACAGAACCTCGAGGA |
| | CTTAGTATGCGTATTATTGGTA | TCTCCGGGTATGACGG |
| M0011 | GAGAACCTGTACTTCCAGAGCGGCC | TCCTTGGAACAGAACCTCGAGAT |
| | ACATTGTCTGGCC | AATGAGTCATACTGTAACACACCG |
| | | C |
| M0012 | GAGAACCTGTACTTCCAGAGCAAAAT | TCCTTGGAACAGAACCTCGAGTTT |
| | GAAGATGAAACTTGCATCGTTC | GACACCTACAGCGTCAAATG |
| M0013 | GAGAACCTGTACTTCCAGAGCAATCC | TCCTTGGAACAGAACCTCGAGGC |
| | TTTGCTTATCTTAACGTTCG | TATTCGCTGCGATTGTG |
| M0014 | GAGAACCTGTACTTCCAGAGCAACAT | TCCTTGGAACAGAACCTCGAGAA |
| | TAGCGCATTTAAGATTGCG | CATTCGGCAACAAATAGTCAC |
| M0015 | GAGAACCTGTACTTCCAGAGCCATCA | TCCTTGGAACAGAACCTCGAGGC |
| | AGTGGGTTGCCCAG | TCCACCCATTTCCTTCG |
| M0016 | GAGAACCTGTACTTCCAGAGCAGCG | TCCTTGGAACAGAACCTCGAGGC |
| | CTTTTGTCGGCGC | TAAAGTTGATTCCACCGTCCAC |
| M0017 | GAGAACCTGTACTTCCAGAGCGCGG | TCCTTGGAACAGAACCTCGAGCA |
| | ATAAAGTTAATGTTTGTATTGTC | TGTGTTCCGGGTGATTAC |
| M0018 | GAGAACCTGTACTTCCAGAGCCGACT | TCCTTGGAACAGAACCTCGAGCG |
| | TACCGTCGAGTTAATTCAAAAC | TTTGCATTTTGTCCCCT |
| M0019 | GAGAACCTGTACTTCCAGAGCAGTG | TCCTTGGAACAGAACCTCGAGGA |
| | GAGTAAATGTGGCTGGAG | ATTGTAATTCTTTTTGGATGCCCC |
| | | A |
| M0020 | GAGAACCTGTACTTCCAGAGCGCAA | TCCTTGGAACAGAACCTCGAGGT |
| | CGGCCACCTCAGC | TAGCTGCAGCGGCTGC |
| M0021 | GAGAACCTGTACTTCCAGAGCGAATT | TCCTTGGAACAGAACCTCGAGCA |
| | CGAGAAAATTAAGGTGATTAATCCC | AACGACTATTATTACCAAGCAAAC |
| M0022 | GAGAACCTGTACTTCCAGAGCGACC | TCCTTGGAACAGAACCTCGAGTG |
| | GTCGCCGCTCTCTG | ATTCCAATTTAGAGACATCGCGTG |
| | | AAG |
| M0023 | GAGAACCTGTACTTCCAGAGCAAGAC | TCCTTGGAACAGAACCTCGAGCT |
| | CATCTTATCAAGCAGCC | GTGATGATGCATAATCCGC |
| M0024 | GAGAACCTGTACTTCCAGAGCGTAGT | TCCTTGGAACAGAACCTCGAGCA |
| | TGTCGCAATGGCAGTG | GCTCATCAACTAAGCGGG |
| M0025 | GAGAACCTGTACTTCCAGAGCACAGT | TCCTTGGAACAGAACCTCGAGGA |
| | TTTGAATTACCTTCGCAC | TCTCTACCATTGCGATCAAG |
| M0026 | GAGAACCTGTACTTCCAGAGCGCTC | TCCTTGGAACAGAACCTCGAGCT |
| | CAATCAAGGTAGGCATT | TAGAAGCATCAACTTTCGCAAC |
| M0027 | GAGAACCTGTACTTCCAGAGCTCCC | TCCTTGGAACAGAACCTCGAGCA |
| | GTTTTAGCGGTTATAATGTC | AGTTTGAGTAGTCACGGGAC |
| M0028 | GAGAACCTGTACTTCCAGAGCACAAC | TCCTTGGAACAGAACCTCGAGGT |
| | GGCGGTACTGGGAG | GTCCTGGAAGTGGCAGC |
| M0029 | GAGAACCTGTACTTCCAGAGCACAG | TCCTTGGAACAGAACCTCGAGCG |
| | CACAGAATACGTTTGACG | CTAAAAAATTGATGAATCCTCCTA |
| | | CTG |
| M0030 | GAGAACCTGTACTTCCAGAGCATCAG | TCCTTGGAACAGAACCTCGAGCC |
| | TTTAATCGCTGCATTGG | GGCGTTCAAGGATTTCG |
| M0031 | GAGAACCTGTACTTCCAGAGCATGGA | TCCTTGGAACAGAACCTCGAGTG |
| | AGAGTGCTACCAGACG | TGCCTGTGTACATACAACG |
| M0032 | GAGAACCTGTACTTCCAGAGCCCAG | TCCTTGGAACAGAACCTCGAGCA |
| | AAAGTATTACGCTGTACTCCG | CCTGTCCACCCACGC |
| M0033 | GAGAACCTGTACTTCCAGAGCGTGTT | TCCTTGGAACAGAACCTCGAGCA |
| | GTACTGCTGCCCG | TTCCATTATAGGCCGGACC |
| M0034 | GAGAACCTGTACTTCCAGAGCCGCG | TCCTTGGAACAGAACCTCGAGTG |
| | CACATCCCGGTGG | GGGGAAAGGATTGGTTCTGC |
| M0035 | GAGAACCTGTACTTCCAGAGCGCAA | TCCTTGGAACAGAACCTCGAGGG |
| | GCCCAAATTTTTTTTTCTTTC | GCAGAAAATTGTTACTTTGG |
| M0036 | GAGAACCTGTACTTCCAGAGCGCAA | TCCTTGGAACAGAACCTCGAGGA |
| | CCCGCGTAATCCCG | ACATCGCAGCTTTCAAGCG |
| M0037 | GAGAACCTGTACTTCCAGAGCGCTG | TCCTTGGAACAGAACCTCGAGTA |
| | CTGCAATCGAGATC | TCTGATCGTCCCACAAATCAG |
| M0038 | GAGAACCTGTACTTCCAGAGCTCTTC | TCCTTGGAACAGAACCTCGAGTT |
| | ATTTTTTAAAAGTTTGTTTTCTAAACG | CCGCCAGGGGACCC |
| | CGAATC | |
| M0039 | GAGAACCTGTACTTCCAGAGCGCGG | TCCTTGGAACAGAACCTCGAGCT |
| | CAGTGGCTTCGGC | CTTTAGATACCAAGGATTTCTTCA |
| | | CACAGTCGAC |
| M0040 | GAGAACCTGTACTTCCAGAGCGCGC | TCCTTGGAACAGAACCTCGAGGA |
| | AAGTGTTAATTGTCGGTG | TGGGAAAGCCGATAGCCATC |
| M0041 | GAGAACCTGTACTTCCAGAGCCCGTT | TCCTTGGAACAGAACCTCGAGAG |
| | AGCTGCTGTGTCTCTTG | ATTTATCGGGTGACGAAGG |
| M0042 | GAGAACCTGTACTTCCAGAGCGTGG | TCCTTGGAACAGAACCTCGAGAG |
| | CTGCCGCAATGTTAC | CGTACGTTGTCACGTATTGC |
| M0043 | GAGAACCTGTACTTCCAGAGCGCGC | TCCTTGGAACAGAACCTCGAGCA |
| | TGGAAAAACACATC | TATAATATTCCGGTTTCAGTACCC |
| M0044 | GAGAACCTGTACTTCCAGAGCGATCC | TCCTTGGAACAGAACCTCGAGCG |
| | ATTATGCACCGCGTC | CATTGCGCTCCCATGG |
| M0045 | GAGAACCTGTACTTCCAGAGCAAAAT | TCCTTGGAACAGAACCTCGAGCG |
| | CAGTCAGTACGCATGCC | CGTTACGTTCCCAGGG |
| M0046 | GAGAACCTGTACTTCCAGAGCCTGCA | TCCTTGGAACAGAACCTCGAGTT |
| | CATCCCTATCTTCC | AGGCGTTACGTTGCCAAG |
| M0047 | GAGAACCTGTACTTCCAGAGCACCGT | TCCTTGGAACAGAACCTCGAGTT |
| | TTCCCAGTATGCCTG | AGGCATTACGTTGCCATGG |
| M0048 | GAGAACCTGTACTTCCAGAGCACCTA | TCCTTGGAACAGAACCTCGAGCA |
| | TACTACACGTCAGATCGG | CACTACCGGAAATGAAAAACCAC |

### Method:

Templates designed as C-terminal sfGFP-fusion proteins were synthesised by a commercial supplier and received as 25 nmol syntheses reconstituted in 20 μL TE buffer (1.25 nmol/μL).

All templates were diluted 0.1× as shown in Table 1.

TABLE 1
Dilution of DNA templates

| Component | μL |
|---|---|
| 1.25 nmol/uL DNA template | 1 |
| MilliQ water | 9 |
| TOTAL (0.125 nmol/uL template) | 10 |

AdaptPCR primer mixes were designed to target the CDS within the template sequence of each of the 48 templates listed. Each of these primers had a universal 5′ tail portion (see table 2) and a template-specific 3′ head portion and were prepared as a ready to use mix. AdaptPCR primer mixes were received as 1 nmol syntheses reconstituted in 100 μl TE buffer.

TABLE 2
AdaptPCR primer mixes (M0001-M0048)

| | |
|---|---|
| PCR FWD 5′ tail sequence | 5′ GAGAACCTGTACTTCCAGAGC |
| PCR_REV 5′ tail sequence | 5′ TCCTTGGAACAGAACCTCGAG |
| 3′ head sequence | Template specific |

Each of the 48× templates was PCR amplified with the corresponding adaptPCR primer mix according to the reaction conditions shown in Table 3, and thermocycling conditions in Table 4.

TABLE 3
AdaptPCR reaction conditions

| Component | μL |
|---|---|
| 0.125 nmol/μL DNA template | 1 |
| 10 μM AdaptPCR primer mix | 1 |
| 2X Q5 Hotstart PCR mastermix | 10 |
| MilliQ water | 8 |
| TOTAL | 20 |

TABLE 4
AdaptPCR thermocycling conditions

| Step | Description | Temperature (° C.) | Time (sec) |
|---|---|---|---|
| 1 | Initial denaturation | 98 | 30 |
| 2 | Denaturation | 98 | 10 |
| 3 | Annealing | 65 | 30 |
| 4 | Extension | 72 | 60 |
| 5 | Go to step 2; repeat 29 cycles | | |
| 6 | Final extension | 72 | 120 |
| 7 | Hold | 4 | ∞ |

Reactions were paused after 10 cycles to remove 5 μL of 10-cycle amplicon. Then the program was resumed and allowed to run a further 20 cycles.

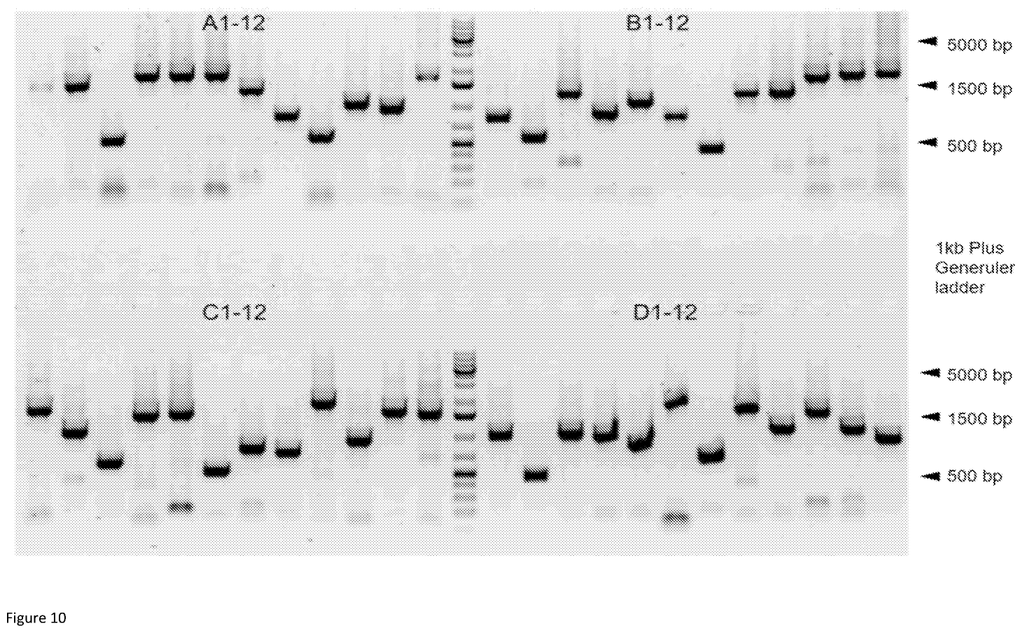

Aliquots of the 30-cycle adaptPCR amplicons were analyzed by 1% TBE agarose gel electrophoresis stained with SybrSafe dye. Gel was run at 100V for 30 minutes and visualized on a transilluminator (FIG. 10).

TABLE 5
Dilution of 10-cycle AdaptPCR amplicon

| Component | μL |
|---|---|
| 10 cycle AdaptPCR amplicon | 1 |
| MilliQ water | 49 |
| TOTAL | 50 |

The 10-cycle adaptPCR amplicons were diluted as shown in Table 5 and used as input into universal megaprimer assembly (UMA) reactions to make UMA-LEC linear expression constructs as shown in Table 6 and thermocycling conditions shown in Table 7. The sequences of the single-stranded left flank- and right flank-megaprimer sequences appended to the AdaptPCR amplicon are given in Table 8 along with a cartoon schematic.

TABLE 6
UMA-LEC assembly reaction conditions

| Component | μL |
|---|---|
| 1/50 dilution 10-cycle AdaptPCR template | 1 |
| 235 nM Left megaprimer (LF001) | 4.5 |
| 235 nM Right megaprimer (RF003) | 4.5 |
| 2X NEB Q5 hotstart PCR mastermix | 10 |
| TOTAL | 20 |

TABLE 7
UMA-LEC-PCR thermocycling conditions

| Step | Description | Temperature (° C.) | Time (sec) |
|---|---|---|---|
| 1 | Initial denaturation | 98 | 30 |
| 2 | Denaturation | 98 | 10 |
| 3 | Annealing | 65 | 30 |
| 4 | Extension | 72 | 60 |
| 5 | Go to step 2; repeat 29 cycles | | |
| 6 | Final extension | 72 | 120 |
| 7 | Hold | 4 | ∞ |

TABLE 8
Megaprimer sequences

| | |
|---|---|
| LF001 | LF001BUF_T7P_RBS_TEV |
| | 5′ACCTGATAAGCACGCTGAGTAAACGAAGATTTTCCTTGCGGAGGTTATCTC |
| | GTATCGGTATTTCCCTATCCAGAGTTTATTACTTACAGAAGACAATAATACGA |
| | CTCACTATAGGGAGACCACAACGGTTTCCCTCTAGAAATAATTTTGTTTAACT |
| | TTAAGAAGGAGATATACATATGGAGAACCTGTACTTCCAGAGC |
| RF003 | 3C_L1_sfGFP_STOPSTOP_TERM_RF001BUF |
| | 5′CTCGAGGTTCTGTTCCAAGGACCTGGATCAGCAGGTTCAAGTGCAAGTGGA |
| | AGCAAAGGTGAAGAACTGTTTACCGGCGTTGTGCCGATTCTGGTGGAACTGG |
| | ATGGCGATGTGAACGGTCACAAATTCAGCGTGCGTGGTGAAGGTGAAGGCG |
| | ATGCCACGATTGGCAAACTGACGCTGAAATTTATCTGCACCACCGGCAAACT |
| | GCCGGTGCCGTGGCCGACGCTGGTGACCACCCTGACCTATGGCGTTCAGTG |
| | TTTTAGTCGCTATCCGGATCACATGAAACGTCACGATTTCTTTAAATCTGCAAT |
| | GCCGGAAGGCTATGTGCAGGAACGTACGATTAGCTTTAAAGATGATGGCAAA |
| | TATAAAACGCGCGCCGTTGTGAAATTTGAAGGCGATACCCTGGTGAACCGCA |
| | TTGAACTGAAAGGCACGGATTTTAAAGAAGATGGCAATATCCTGGGCCATAA |
| | ACTGGAATACAACTTTAATAGCCATAATGTTTATATTACGGCGGATAAACAGA |
| | AAAATGGCATCAAAGCGAATTTTACCGTTCGCCATAACGTTGAAGATGGCAGT |
| | GTGCAGCTGGCAGATCATTATCAGCAGAATACCCCGATTGGTGATGGTCCGG |
| | TGCTGCTGCCGGATAATCATTATCTGAGCACGCAGACCGTTCTGTCTAAAGA |
| | TCCGAACGAAAAAGGCACGCGGGACCACATGGTTCTGCACGAATATGTGAAT |
| | GCGGCAGGTATTACGTAATAACTAGCATAACCCCTTGGGGCCTCTAAACGGG |
| | TCTTGAGGGGTTTTTTGTGGAACAGAAGGGTGTCTACCTCTTATTATCGTATC |
| | AACAACGTCTCAGTATGATAGAATCTCAATAAGTTCAGTTTCACAGCCTCGTG |
| | TAAATAGGG |

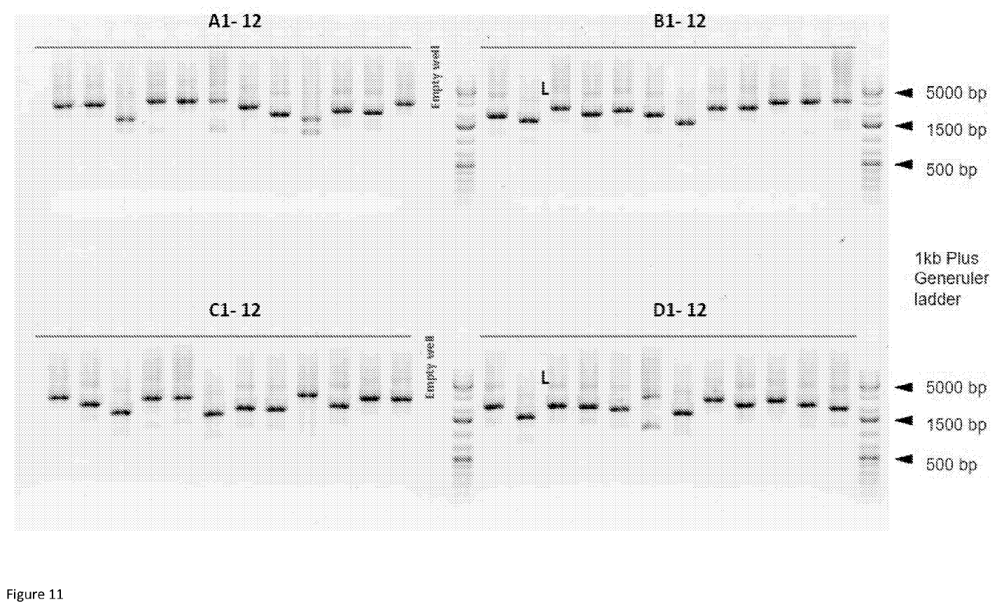

Aliquots of the 30-cycle UMA-LEC-PCR amplicons were analyzed by 1% TBE agarose gel electrophoresis stained with SybrSafe dye. Gel was run at 100 V for 30 minutes and visualized on a transilluminator (FIG. 11).

UMA-LEC-PCR amplicons were purified by GeneJET PCR clean-up columns and eluted in 20 μL EB. These were used directly as expression constructs in LS70 lysate CFPS reactions as shown in Table 9.

TABLE 9
LS70 CFPS reaction conditions

| Component | μL |
|---|---|
| Purified UMA-LEC (4.5 nM) | 2.5 |
| Arbor Biosciences LS70 CFPS mastermix | 9 |
| 2.4 nM p70a_T7rnap_HP | 0.5 |
| TOTAL | 20 |

Reactions were mixed by flicking tubes, centrifuged for 10 sec and then incubated in a static incubator at 29° C. for 18 hours. Expression was first qualitatively assessed by eye as all proteins were sfGFP fusions, and positive expression was observed as a color change from colorless CFPS starting reaction to green/yellow expressed sfGFP-fusion protein.

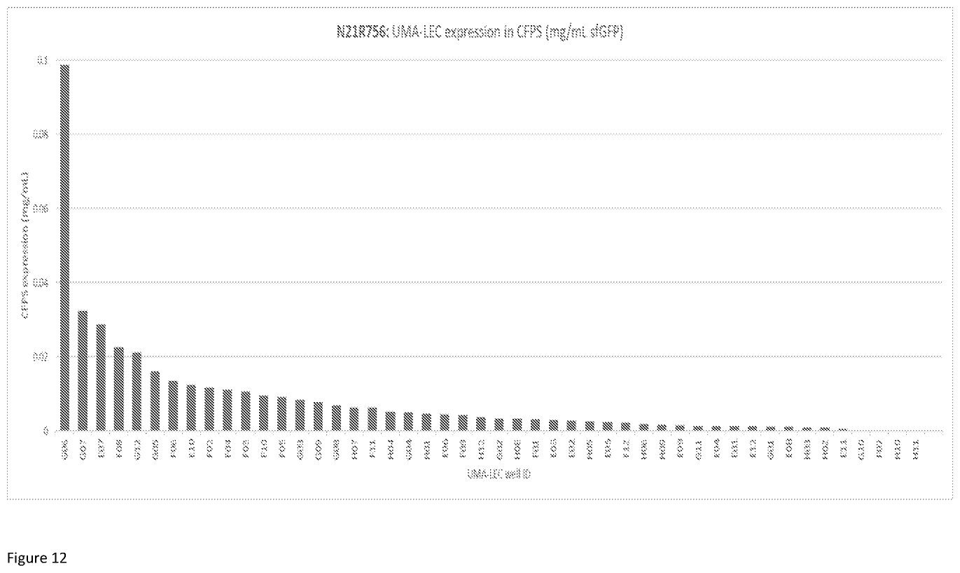

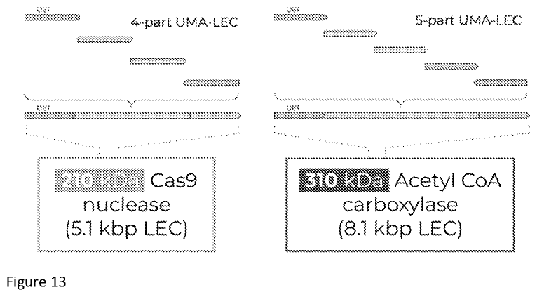

Expression was quantified by fluorimetry. Overnight CFPS reactions were diluted 1/50 in TNG buffer. Dilutions (50 μL per well) were imaged in a 384 well black Corning microtitre plate on a BMG FLUOstar fluorimeter. A ranked expression histogram of the 48 CFPS expressed proteins is shown in FIG. 12.

### Multi-Part Assembly and Activity of a Cas9 Protein

Multi-part amplification is performed using sequences as shown:

| | | |
|---|---|---|
| Cas9_A | 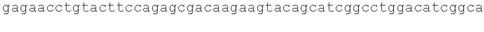 | 2240 |
| | ccaactctgtgggctgggccgtgatcaccgacgagtacaaggtgcccagcaagaa | |
| | attcaaggtgctgggcaacaccgaccggcacagcatcaagaagaacctgatcgga | |
| | gccctgctgttcgacagcggcgaaacagccgaggccacccggctgaagagaacc | |
| | gccagaagaagatacaccagacggaagaaccggatctgctatctgcaagagatct | |
| | tcagcaacgagatggccaaggtggacgacagcttcttccacagactggaagagtcc | |
| | ttcctggtggaagaggataagaagcacgagcggcaccccatcttcggcaacatcgt | |
| | ggacgaggtggcctaccacgagaagtaccccaccatctaccacctgagaaagaa | |
| | actggtggacagcaccgacaaggccgacctgcggctgatctatctggccctggccc | |
| | acatgatcaagttccggggccacttcctgatcgagggcgacctgaaccccgacaac | |
| | agcgacgtggacaagctgttcatccagctggtgcagacctacaaccagcctgttcgag | |
| | gaaaaccccatcaacgccagcggcgtggacgccaaggccatcctgtctgccagac | |
| | tgagcaagagcagacggctggaaaatctgatcgcccagctgcccggcgagaaga | |
| | agaatggcctgttcggaaacctgattgccctgagcctgggcctgacccccaacttcaa | |
| | gagcaacttcgacctggccgaggatgccaaactgcagctgagcaaggacacctac | |
| | gacgacgacctggacaacctgctggcccagatcggcgaccagtacgccgacctgtt | |
| | tctggccgccaagaacctgtccgacgccatcctgctgagcgacatcctgagagtgaa | |
| | caccgagatcaccaaggcccccctgagcgcctctatgatcaagagatacgacgag | |
| | caccaccaggacctgaccctgctgaaagctctcgtgcggcagcagctgcctgagaa | |
| | gtacaaagagattttcttcgaccagagcaagaacggctacgccggctacattgacgg | |
| | cggagccagccaggaagagttctacaagttcatcaagcccatcctggaaaagatgg | |
| | acggcaccgaggaactgctcgtgaagctgaacagagaggacctgctgcggaagc | |
| | agcggaccttcgacaacggcagcatcccccaccagatccacctgggagagctgca | |
| | cgccattctgcggcggcaggaagatttttacccattcctgaaggacaaccgggaaaa | |
| | gatcgagaagatcctgaccttccgcatcccctactacgtgggccctctggccagggg | |
| | aaacagcagattcgcctggatgaccagaaagagcgaggaaaccatcaccccctg | |
| | gaacttcgaggaagtggtggacaagggcgcttccgcccagagcttcatcgagcgga | |
| | tgaccaacttcgataagaacctgcccaacgagaaggtgctgcccaagcacagcct | |
| | gctgtacgagtacttcaccgtgtataacgagctgaccaaagtgaaatacgtgaccga | |
| | gggaatgagaaagcccgccttcctgagcggcgagcagaaaaaggccatcgtgga | |
| | cctgctgttcaagaccaaccggaaagtgaccgtgaagcagctgaaagaggactact | |
| | tcaagaaaatcgagtgcttcgactccgtggaaatctccggcgtggaagatcggttca | |
| | acgcctccctgggcacataccacgatctgctgaaaattatcaaggacaaggacttcc | |
| | tggacaatgaggaaaacgaggacattctggaagatatcgtgctgaccctgacactgt | |
| | ttgaggacagagagatgatcgaggaacggctgaaaacctatgcccacctgttcgac | |
| | gacaaagtgatgaagcagctgaagcggcggagatacaccggctggggcaggctg | |
| | agccggaagctgatcaacggcatccgggacaagcagtccggcaagacaatcctg | |
| | gatttcctgaagtccgacggcttcgccaacagaaacttcatgcagctgatccacgac | |
| | gacagcctgacctttaaagaggacatccagaaagcccaggtgtccggccagggcg | |
| | 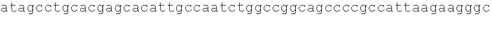 | |
| | 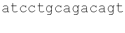 | |
| Cas9_B | 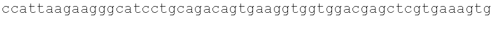 | 1931 |
| | atgggccggcacaagcccgagaacatcgtgatcgaaatggccagagagaaccag | |
| | accacccagaagggacagaagaacagccgcgagagaatgaagcggatcgaag | |
| | agggcatcaaagagctgggcagccagatcctgaaagaacaccccgtggaaaaca | |
| | cccagctgcagaacgagaagctgtacctgtactacctgcagaatgggcgggatatgt | |
| | acgtggaccaggaactggacatcaaccggctgtccgactacgatgtggaccatatc | |
| | gtgcctcagagctttctgaaggacgactccatcgacaacaaggtgctgaccagaag | |
| | cgacaagaaccggggcaagagcgacaacgtgccctccgaagaggtcgtgaaga | |
| | agatgaagaactactggcggcagctgctgaacgccaagctgattacccagagaaa | |
| | gttcgacaatctgaccaaggccgagagaggcggcctgagcgaactggataaggcc | |
| | ggcttcatcaagagacagctggtggaaacccggcagatcacaaagcacgtggcac | |
| | agatcctggactcccggatgaacactaagtacgacgagaatgacaagctgatccgg | |
| | gaagtgaaagtgatcaccctgaagtccaagctggtgtccgatttccggaaggatttcc | |
| | agttttacaaagtgcgcgagatcaacaactaccaccacgcccacgacgcctacctg | |
| | aacgccgtcgtgggaaccgccctgatcaaaaagtaccctaagctggaaagcgagtt | |
| | cgtgtacggcgactacaaggtgtacgacgtgcggaagatgatcgccaagagcgag | |
| | caggaaatcggcaaggctaccgccaagtacttcttctacagcaacatcatgaacttttt | |
| | caagaccgagattaccctggccaacggcgagatccggaagcggcctctgatcgag | |
| | acaaacggcgaaaccggggagatcgtgtgggataagggccgggattttgccaccg | |
| | tgcggaaagtgctgagcatgccccaagtgaatatcgtgaaaaagaccgaggtgca | |
| | gacaggcggcttcagcaaagagtctatcctgcccaagaggaacagcgataagctg | |
| | atcgccagaaagaaggactgggaccctaagaagtacggcggcttcgacagcccc | |
| | accgtggcctattctgtgctggtggtggccaaagtggaaaagggcaagtccaagaa | |
| | actgaagagtgtgaaagagctgctggggatcaccatcatggaaagaagcagcttcg | |
| | agaagaatcccatcgactttctggaagccaagggctacaaagaagtgaaaaagga | |
| | cctgatcatcaagctgcctaagtactccctgttcgagctggaaaacggccggaagag | |
| | aatgctggcctctgccggcgaacgtcagaagggaaacgaactggccctgccctcca | |
| | aatatgtgaacttcctgtacctggccagccactatgagaagctgaagggctcccccg | |
| | aggataatgagcagaaacagctgtttgtggaacagcacaagcactacctggacga | |
| | gatcatcgagcagatcagcgagttctccaagagagtgatcctggccgacgctaatct | |
| | ggacaaagtgctgtccgcctacaacaagcaccgggataagcccatcagagagca | |
| | ggccgagaatatcatccacctgtttaccctgaccaatctgggagcccctgccgccttc | |
| | aagtactttgacaccaccatcgaccggaagaggtacaccagcaccaaagaggtgc | |
| | tggacgccaccctgatccaccagagcatcaccggcctgtacgagacacggatcga | |
| | 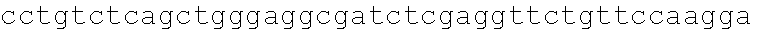 | |

The 3′ end of region A is complementary to 5′ end of the region B (highlighted above). Amplification was performed in one pot using left and right primer sequences below:

Flank 2:

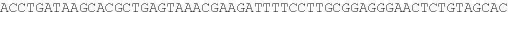

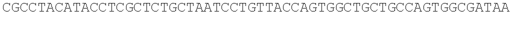

GTCGTGTCTTACCGGGTTGGACTCAAGACGATAGTTACCGGATAAGGCGCAGCGGTC
GGGCTGAACGGGGGGTTCGTGCACACAGCCCAGCTTGGAGCGAACGACCTACACCG
AACTGAGATACCTACAGCGTGAGCTATGAGAAAGCGCCACGCTTCCCGAAGGGAGAA
AGGCGGACAGGTATCCGGTAAGCGGCAGGGTCGGAACAGGAGAGCGCACGAGGGA
GCTTCCAGGGGGAAACGCCTGGTATCTTTATAGTCCTGTCGGGTTTCGCCACCTCTGA
CTTGAGCGTCGATTTTTGTGATGCTCGTCAGGGGGGCGGAGCCTATGGAAAAACGCC
AGCAACGCGATCCCGCGAAATTAATACGACTCACTATAGGGAGACCACAACGGTTTCC

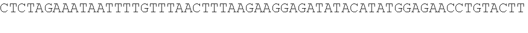

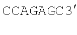

Flank 352

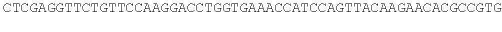

GCCAAATATTTCACCGAATAATAATTGATTGACTAGCATAACCCCTTGGGGCCTCTAA
CGGGTCTTGAGGGGTTTTTTGCTGAAAGCCAATTCTGATTAGAAAAACTCATCGAGCA
TCAAATGAAACTGCAATTTATTCATATCAGGATTATCAATACCATATTTTTGAAAAAGCC
GTTTCTGTAATGAAGGAGAAAACTCACCGAGGCAGTTCCATAGGATGGCAAGATCCTG

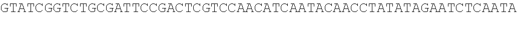

AGTTCAGTTTCACAGCCTCGTGTAAATAGGG
In the presence of terminal amplification primers

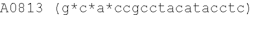

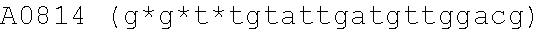

### Using PHIRE Hotstart Polymerase and the Following Cycle:

PCR PROGRAM

| Step | Temp | Time (sec) | | |
|---|---|---|---|---|
| Denaturation | 95° C. | 2 | min | |
| Denaturation | 95° C. | 20 | sec | 24 cycles |
| Annealing | 62° C. | 20 | sec | |
| Extension | 72° C. | 115 | sec | |
| Extension | 72° C. | 2 | min | |

| | | |
|---|---|---|
| Hold | 12° C. | Infinite |

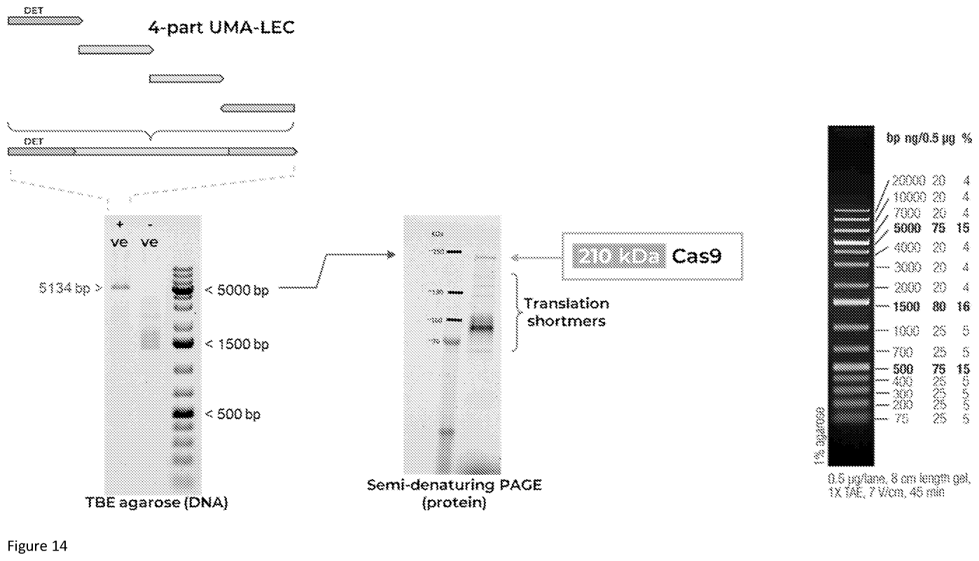

The resultant amplicon was run on a 1% agarose gel, shown in FIG. 14.

The PCR step can be repeated using terminal primers to obtain more full-length construct.

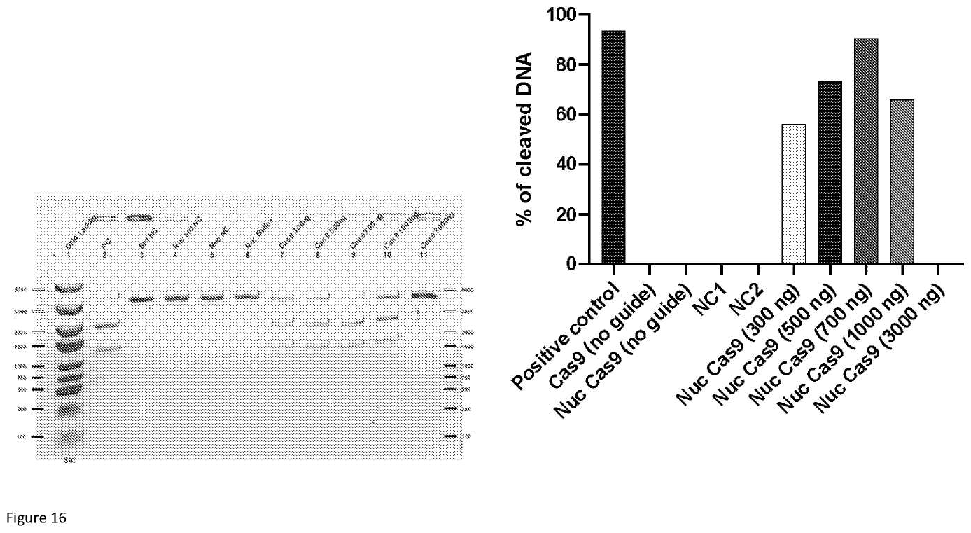

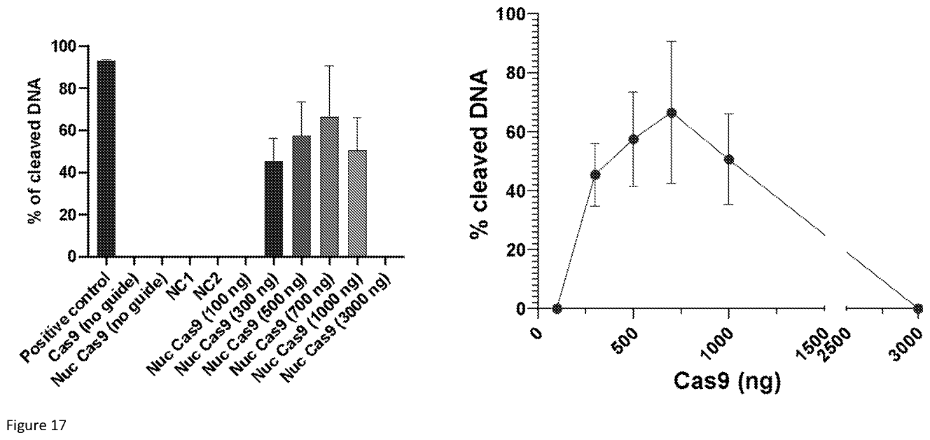

Amplicons can be used to express Cas9 using a reconstituted cell-free expression system. Expression of the 210 kDa protein is shown in FIG. 14. Where the sequences express a strep-tag, the protein can be isolated using Strep-Tactin® beads, and eluted using Strep-tactin®XT Elution Buffer. After elution the activity was determined using a Cas9 activity assay looking at DNA cleavage. Results from the cleavage assay are shown in FIGS. 16 and 17. DNA strand cleavage can be seen in proportion to the Cas9 concentration. At the highest concentration (3000 ng) excess Cas9 causes aggression of DNA target, resulting in no cleavage. The same amount of target DNA is used per reaction (100 ng). Cleaved products have expected molecular weight.

Multi-part assembly of an 8 kb construct to produce a 310 kDa Acetyl CoA carboxylase Multi-part amplification is performed using sequences as shown:

| | | |
|---|---|---|
| Insert A | 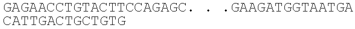 | 2400 |
| Insert B | 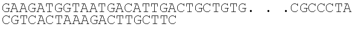 | 2396 |
| Insert C | 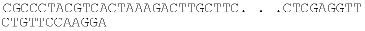 | 2336 |

The 3′ end of region A is complementary to 5′ end of the region B (highlighted above). The 3′ end of region B is complementary to 5′ end of the region C (highlighted above). Amplification was performed in one pot using left and right primer sequences below:

Flank 2:

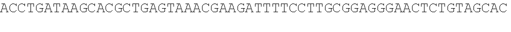

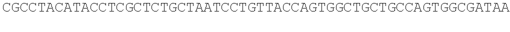

GTCGTGTCTTACCGGGTTGGACTCAAGACGATAGTTACCGGATAAGGCGCAGCGGTC
GGGCTGAACGGGGGGTTCGTGCACACAGCCCAGCTTGGAGCGAACGACCTACACCG
AACTGAGATACCTACAGCGTGAGCTATGAGAAAGCGCCACGCTTCCCGAAGGGAGAA
AGGCGGACAGGTATCCGGTAAGCGGCAGGGTCGGAACAGGAGAGCGCACGAGGGA
GCTTCCAGGGGGAAACGCCTGGTATCTTTATAGTCCTGTCGGGTTTCGCCACCTCTGA
CTTGAGCGTCGATTTTTGTGATGCTCGTCAGGGGGGCGGAGCCTATGGAAAAACGCC
AGCAACGCGATCCCGCGAAATTAATACGACTCACTATAGGGAGACCACAACGGTTTCC

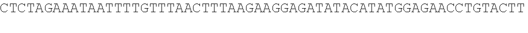

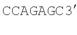

Flank 352

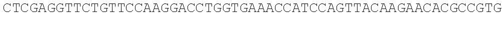

GCCAAATATTTCACCGAATAATAATTGATTGACTAGCATAACCCCTTGGGGCCTCTAAA
CGGGTCTTGAGGGGTTTTTTGCTGAAAGCCAATTCTGATTAGAAAAACTCATCGAGCA
TCAAATGAAACTGCAATTTATTCATATCAGGATTATCAATACCATATTTTTGAAAAAGCC
GTTTCTGTAATGAAGGAGAAAACTCACCGAGGCAGTTCCATAGGATGGCAAGATCCTG

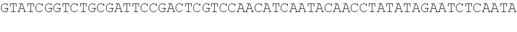

AGTTCAGTTTCACAGCCTCGTGTAAATAGGG
In the presence of terminal primers

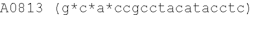

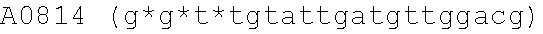

### Using PHIRE Hotstart Polymerase and the Following Cycle:

PCR PROGRAM

| Step | Temp | Time (sec) | | |
|---|---|---|---|---|
| Denaturation | 95° C. | 2 | min | |
| Denaturation | 95° C. | 20 | sec | 24 cycles |
| Annealing | 62° C. | 20 | sec | |
| Extension | 72° C. | 170 | sec | |
| Extension | 72° C. | 2 | min | |

| | | |
|---|---|---|
| Hold | 12° C. | Infinite |

The PCR step can be repeated using terminal primers to obtain more full-length construct.

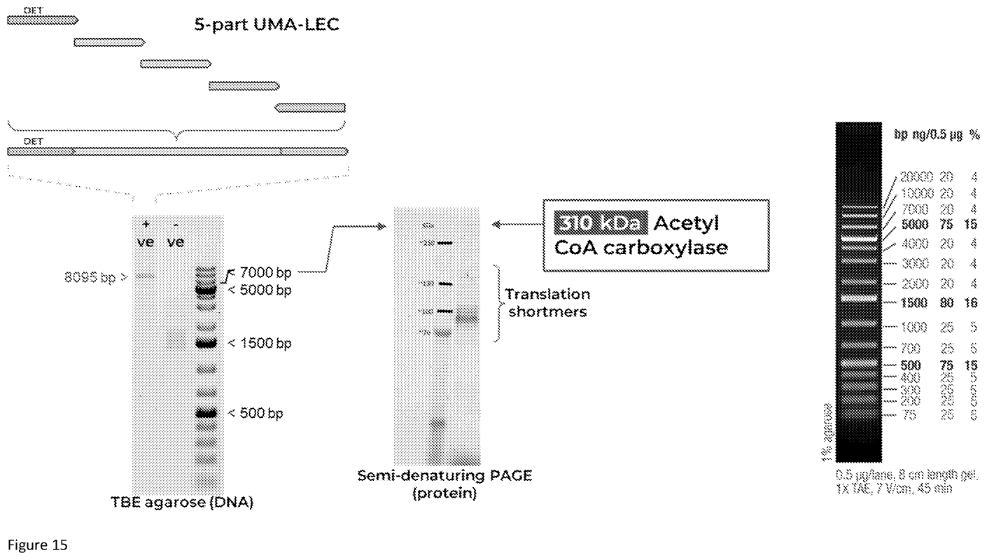

Amplicons can be used to express the 310 kDa Acetyl COA carboxylase using a reconstituted cell-free expression system. Expression of the 310 kDa protein is shown in FIG. 15.

### Optimising PCR Cycle Numbers for Protein Expression Constructs

More PCR cycles gives a greater mass of product, but appears to increase the ratio of short extension products. Using a protocol with 35 PCR cycles, increased amounts of truncated protein products were detected in the CFPS mixtures even when the detector tag was on the C-terminus. Certain flank primers that presented these issues were and tested with both 80 nM and 20 nM concentrations using a different number of PCR cycles was tested in order to identify whether the truncated products are originating from the assembly process.

### Methods and Results

### Flank Primers Tested

| SOL | | | |
|---|---|---|---|
| NAME | DET_SOL | Left flank design | Right flank design |
| P17 | CSOL - | LBUF400_T7P_RBS2_START_E18_{——} | TEV_L25_P17_L22_CCGFP11V1_{——} |
| | CDET | STREPV4_L2_{——}3C | STOP_TERM_RBUF200 |
| MOCR | NSOL - | LBUF400_T7P_RBS2_START_E18_MOCR_{—} | TEV_{——}L22_CCGFP11V1_{——}STOP_{—} |
| | CDET | L19_STREPV4_L2_{——}3C | TERM_RBUF200 |
| MOCR | CSOL - | LBUF400_T7P_RBS2_START_E18_{———} | TEV_L25_MOCR_{——}L21_STREPV4_{—} |
| | NDET | CCGFP11V1_L1_3C | STOP_TERM_RBUF200 |
| GST | NSOL - | LBUF400_T7P_RBS2_START_E18_GST_{—} | TEV_{——}L22_CCGFP11V1_L21_{—} |
| | CDET | L19_{———}3C | STREPV4_STOP_TERM_RBUF200 |

### Inserts Tested

| | length | |
|---|---|---|
| oid51 | 2124 | extension time 50 s |
| oid246 | 1383 | extension time 40 s |

| | | | | |
|---|---|---|---|---|
| 80 nM | 28 cycles | 1 well | x 4 flanks | x 2 inserts |
| 20 nM | 28 cycles | 2 wells | | |
| 20 nM | 30 cycles | 2 wells | | |
| 20 nM | 32 cycles | 2 wells | | |
| 20 nM | 35 cycles | 1 well | | |
| | | 8 wells | 64 | PCR samples |
| | | | 40 | exp samples |

80 nM MP PCR

| Component | Vol (μL) |
|---|---|
| PHIRE Hotstart (2x) | 25 |
| Left MP (80 nM) | 1 |
| Right MP (80 nM) | 1 |
| A0813 (10 μM) | 1.5 |
| A0814 (10 μM) | 1.5 |
| Template 2 nM | 1 |
| NF water | 19 |
| Total | 50 |

20 nM MP PCR

| | |
|---|---|
| Template 2 nM | 1 |
| Left MP 8 nM | 2.5 |
| Right MP 8 nM | 2.5 |
| A0813 (10 μM) | 1.8 |
| A0814 (10 μM) | 1.8 |
| NF water | 20.4 |
| Polymerase | 30 |
| Total | 60 |

PCR PROGRAM

| Step | Temp | Time (sec) | |
|---|---|---|---|
| Initial Denaturation | 95 | 120 | |
| Denaturation | 95 | 20 | X Cycles |
| Annealing | 63 | 20 | |
| Extension | 72 | 40 | |
| Final Extension | 72 | 120 | |
| Hold | 10 | ∞ | |

X = 27, 29, 31, 34

Gel samples were prepared before the purification and they were loaded on 1% agarose gel (100 V, 40 min) to confirm full length products were obtained.

### DNA Purification (Commercial Protocol)

### NUC Plate

Transferred 60 μL of Nuclease free water and 120 μL of NUC pure plus and then added 60 μL of the PCR mix into the NUC plate (for 1-well reactions)

Alternatively, transferred 120 μL of NUC pure plus and then added 2×60 μL of the PCR mix into the NUC plate (for 2-well reactions)

### EtOH Plates (×2)

Used the 1200 μL multichannel pipette to load 400 μL (3×400 μL multi-dispense) of freshly made 80% EtOH

### Elution Plate

50 μL of 10 nM HEPES containing 0.05% F-127

### Qubit DNA Quantification (Commercial Protocol)

All samples were diluted 1:50 (98 μL of 1×TE+2 μL of DNA)—the plate was covered and spinned.

Transferred 198 μL of 1×dsDNA HS working solution to the wells of a 96-well microplate and added 2 μL of the diluted samples using the multichannel pipette.

The plate was covered, mixed and spinned and incubated at rt for 10 min before taking the fluorometer measurement.

Average concentration values were normalized to 1 well of 60 μL and the data are shown in the table below.

| Conditions | Average Norm nM |
|---|---|
| 80 nM 28 cycles | 57.54 |
| 20 nM 28 cycles | 49.99 |
| 20 nM 30 cycles | 55.51 |
| 20 nM 32 cycles | 70.56 |
| 20 nM 35 cycles | 85.41 |

All samples were then normalized to 24 nM in order to be used for CFPS tests.

### CFPS

All normalized samples were used for CFPS expression (4 μL of reconstituted expression reagent+1 μL of DNA 24 nM, incubation for 4 h at 28 C.).

ccGFP1-10 detector protein was added (1 μL) and the plate was incubated for another 5 h at 28 C.

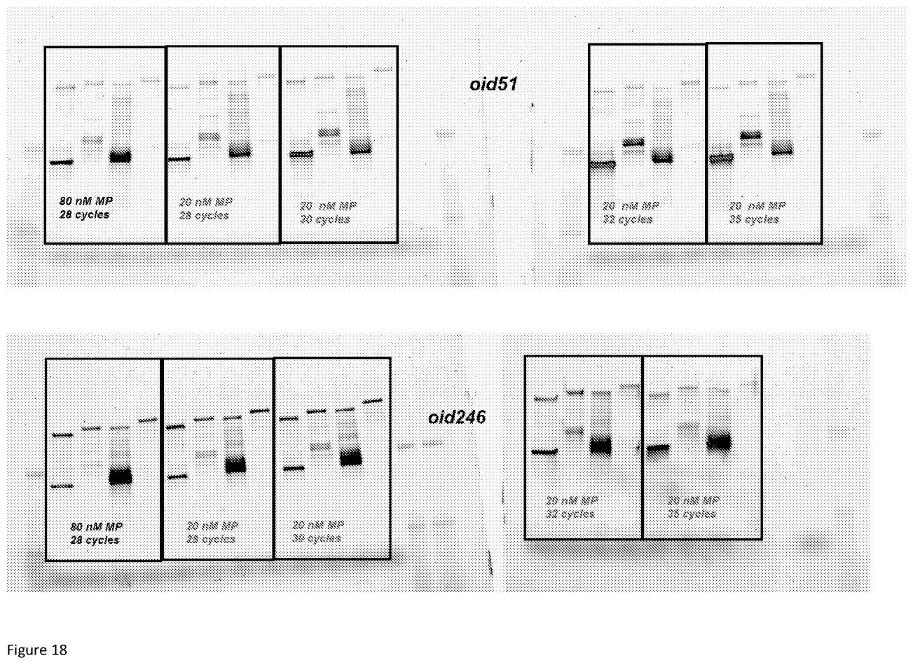

Semi-native PAGE gels are show in FIG. 18. Truncated products exist for both concentrations. No difference observed between a 4-fold concentration difference. The amount of truncated products in the CFPS mixture is increasing with the increase of the PCR cycles. The 4 flank primers shown indicate that for lane 3 (NDet) the amount of detected short product is high as the flank is detected. Even for C-terminal detectors, where the insert is needed for successful amplification and detection, short products are increasing with greater cycle number. Thus 28 cycles gives the optimal balance of DNA obtained vs correct expression. Fewer cycles gives insufficient template. Higher cycles give more incorrect extension.

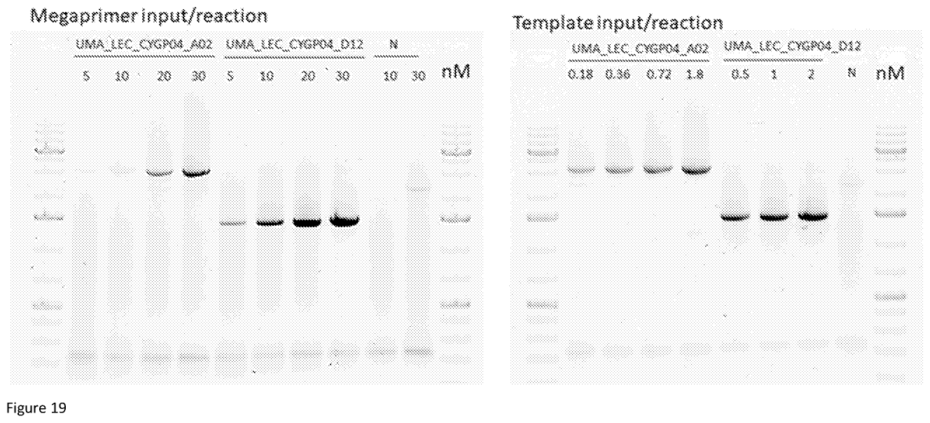

The rations of input concentrations and primers was evaluated, data shown in FIG. 19. The data shows that below 20 nM concentration of the left and right flank primers, little amplicon is seen. It can be seen that the amplicon concentration gradually increases with the increase in template concentration and with primer concentration.

The PCR conditions and ratios are as shown below:

| Final | Final | Final | No | | | | RATIO | | | |
|---|---|---|---|---|---|---|---|---|---|---|
| MP | POI | Primer | of | Yield | Construct | Yield | Final | Final | Final | |
| (nM) | (nM) | (nM) | cycles | (ng/μl) | length | nM | MP | POI | Primer | Result |
| 0.333 | 0.0333 | 300,000 | 28 | | | | 10.000 | 1.000 | 10000.000 | optimised |
| | | | | | | | | | | protocol, |
| | | | | | | | | | | no smear |
| | | | | | | | | | | appears, |
| | | | | | | | | | | concentration |
| | | | | | | | | | | in |
| | | | | | | | | | | expected |
| | | | | | | | | | | range of |
| | | | | | | | | | | 40-60 ng/μl |
| 0.4 | 0.01722 | 500 | 30 | 109 | 3300 | 50.05 | 23.2 | 1 | 29040 | varying |
| 0.4 | 0.03444 | 500 | 30 | 106 | 3300 | 48.82 | 11.6 | 1 | 14520 | template |
| 0.4 | 0.06887 | 500 | 30 | 120 | 3300 | 55.25 | 5.8 | 1 | 7260 | conc, on |
| | | | | | | | | | | the gel, we |
| | | | | | | | | | | can see |
| | | | | | | | | | | that the |
| | | | | | | | | | | conc |
| | | | | | | | | | | gradually |
| | | | | | | | | | | increases |
| | | | | | | | | | | with the |
| | | | | | | | | | | increase in |
| | | | | | | | | | | template |
| | | | | | | | | | | conc, |
| 0.1 | 0.00360 | 500 | 30 | 80 | 3300 | 36.73 | 27.8 | 1 | 138889 | Increasing |
| 0.2 | 0.00360 | 500 | 30 | 86 | 3300 | 39.33 | 55.6 | 1 | 138889 | concentration |
| | | | | | | | | | | of |
| | | | | | | | | | | megaprimer |
| | | | | | | | | | | led to |
| | | | | | | | | | | increasing |
| | | | | | | | | | | concentration |
| | | | | | | | | | | of the |
| | | | | | | | | | | amplicon |
| | | | | | | | | | | band |

The optimised ration requires a large excess of the amplification primer in order to obtain sufficient material. Having a high level of the flank primers leads to having flank primers remaining which give shortened extension products.

### Flank Sequence Optimisation

The flank design was examined to identify the best solubility tags and the best positions of the variety of elements for solubility tags, detection tags and purification tags. Left and right flanks having various elements were studied. The solubility tags were selected from:

| ID | Tag |
|---|---|
| SOL_01 | P17 |
| SOL_02 | NEXT |
| SOL_03 | Fh8 |
| SOL_04 | SUMO-3 |
| SOL_05 | Trx |
| SOL_06 | Mocr |
| SOL_07 | SNUT |
| SOL_8 | GST |
| SOL_9 | MBP |
| SOL_10 | CusF |
| SOL_11 | ZZ |

95 combinations of left flanks and right flanks were amplified against a variety of 8 insert sequences using 35 cycles of PCR. The PCR products used for CFPS, run on a gel and characterised as below:

### LEGEND

Score Description
0 Only desired band
2 One additional band
10 Several more bands

The results are tabulated below.

Based on this information, the following flanks were evaluated as:
SOL-POI-DET-PUR Good
PUR-SOL-POI-DET Good
POI-SOL-DET Bad as SOL at the POI C term was not desirable.
POI-DET-PUR Good (control; needs SOL for usage)
PUR-POI-DET Bad as high frequency of shortmers >60%
POI-DET (Control only, needs PUR and SOL)

Therefore the panel taken forward was
PUR-SOL-POI-DET
SOL-POI-DET-PUR
POI-DET-PUR
POI-DET

It is clear that the amplification process is flank sequence and concentration dependent and that not all flanks behave equally. Flanks having a detector tag on the C terminus and the solubility tag on the N terminus were advantageous for the production and detection of full length expression constructs. Certain common solubility tags such as MOCR, NEXT and GST behaved poorly for expressing constructs. 22 constructs were further tested as shown below:

Templates giving multiple expression bands were removed, therefore the best performing and constructs were chosen for further use.

A panel of 16 different inserts was screened against 22 flanks to measure 352 separate protein expression conditions. The process reliably generates high quality amplicon constructs on a diverse set of POI (n=16) at the correct target yield in 28 cycles of PCR:
93% Grade 1 constructs
100% constructs yield 720 nmol=30 μL 24 nM LEC

Expression conditions identified that the majority of constructs express solubly and can be purified from either e Coli cellular lysate or reconstituted systems.

| | lysate | reconstituted |
|---|---|---|
| % expressed constructs | 90.1 | 96.3 |
| % solubly expressed constructs* | 79.3 | 82.4 |
| % expressed constructs purified** | 87.9 | 88.1 |

Panel Architecture

### SOL-POI-DET-PUR ( 8/12=66.6%)

### SOL-PUR-POI-DET ( 4/12=33.3%)·

7 unique choice of SOL tags: P17, CUSF, ZZ, SUMO, TRX, FH8, SNUT
4 SOL tags represented with both N and C term Strep tag.
‘No SOL’ forms a part of the panel
MOCR, NEXT and GST underperformed in both LUPA and PF2.1
   Optimum Panel Identified

1. A method of providing a variety of nucleic acid expression constructs suitable for cell-free protein expression, wherein the method comprises:
i. taking one or more double stranded target nucleic acids, one of the nucleic acids having an end A0 and one having an end B0, wherein A0 and B0 are either connected directly in a single double stranded sequence or can be connected via hybridisation of multiple strands;
ii. amplifying the target nucleic acid with multiple left flank primers and one or more right flank primers to produce a population of constructs having different solubility tags or ribosome binding sites, wherein:
each left flank primer comprises at least a promoter sequence, a sequence encoding for a ribosome binding site for a particular species, an optional solubility tag and, at its 3′ end, a sequence complementary to A0;
and the right flank primer comprises a detection tag, an optional solubility tag, a terminator sequence, a sequence encoding for a stop codon and, at its 3′ end, a sequence complementary to B0;
iii. amplifying the products produced having the left and right flanks using amplification primers complementary to the left and right flanks to selectively amplify the full-length constructs and reduce the proportion of residual left flank primers, wherein the amplification uses at least 100 fold concentration of amplification primers in proportion to the flanking primers;
to produce a population of linear double-stranded expression constructs having a variety of solubility tags or ribosome binding sites suitable for cell-free protein expression of proteins which can be detected.

2. The method according to claim 1, wherein a population of expression constructs having different ribosome binding sites or 5′-UTR's is formed in a single composition.

3. The method according to claim 1, wherein the variety of nucleic acid expression constructs is separate and separate members the population contain different solubility tags on either the N or C side of target sequence.

4. The method of providing a nucleic acid expression construct suitable for cell-free protein expression according to any one of claims 1 to 3, wherein the method comprises amplifying a starting nucleic acid sequence with a forward adapter primer and a reverse adapter primer wherein:
the forward adapter primer comprises at its 3′ end a matching sequence A1 which can bind to a first region of the nucleic acid sequence, and at its 5′ end a sequence A0;
and the reverse adapter primer comprises at its 3′ end a matching sequence B1 which can bind to a second region of the nucleic acid sequence, and at its 5′ end a sequence B0;
to produce the double-stranded target nucleic acid sequence having ends A0 and B0.

5. The method according to claim 4 wherein the amplification to introduce ends A0 and B0 is performed in a single amplification also using the left and right flank primers and the terminal amplification primers to produce the nucleic acid expression constructs.

6. The method according to claim 4 or claim 5, wherein each of the matching sequences A1 and B1 are independently between 10 and 50 nucleotides in length.

7. The method according to any one of claims 1 to 6, wherein the method uses a first nucleic acid having an end A0 and an end C1, and a second nucleic acid having an end B0 and end C1′, wherein C1 and C1′ are complementary, to produce a multi-part extension product having A0 and B0 using two shorter extension products.

8. The method according to any one of claims 1 to 7, wherein A0 and/or B0 encode for protease cleavage sites in an expressed amino acid sequence.

9. The method according to claim 8, wherein the protease is selected from TEV, C3, EK, FXA, FN or Thrombin.

10. The method according to any one of claims 1 to 9, wherein each left flank primer comprises a different sequence encoding for ribosome interaction sites selected from alternative ribosome binding sites or internal ribosome entry sites.

11. The method according to any one of claims 1 to 10, wherein the detection tags are components of fluorescent proteins.

12. The method according to any one of claims 1 to 11, wherein the left or right flank primer comprises a purification tag selected from:
Alfa-tag (SRLEEELRRRLTE)
Avi-tag (GLNDIFEAQKIEWHE)
C-tag (EPEA)
Calmodulin-tag (KRRWKKNFIAVSAANRFKKISSSGAL)
Dogtag (DIPATYEFTDGKHYITNEPIPPK)
E-tag (GAPVPYPDPLEPR)
FLAG (DYKDDDDK)
G4T (EELLSKNYHLENEVARLKK)
HA (YPYDVPDYA)
His (HHHHHH)
Isopeptag (TDKDMTITFTNKKDAE)
lanthanide binding tag (LBT)
(FIDTNNDGWIEGDELLLEEG)
Myc (EQKLISEEDL)
NE-Tag (TKENPRSNQEESYDDNES)
Poly Glutamate-tag (EEEEEEE)
Poly Arginine-tag (RRRRRRR)
Rho1D4-tag (TETSQVAPA)
SBP-tag (MDEKTTGWRGGHVVEGLAGELEQLRARLEHHPQGQREP)
Sdytag (DPIVMIDNDKPIT)
SH3 (STVPVAPPRRRRG)
SNAC (GSHHW)
Snooptag (KLGDIEFIKVNK)
Softag 1 (SLAELLNAGLGGS)
Softag 3 (TQDPSRVG)
Spot-tag (PDRVRAVSHWSS)
Spytag (AHIVMVDAYKPTK)
S-tag (KETAAAKFERQHMDS)
Strep-tag (AWAHPQPGG) (AWRHPQFGG)
Strep-tag II (WSHPQFEK)
T7tag (MASMTGGQQMG)
TC-tag (EVHTNQDPLD)
Ty-tag (CCPGCC)
VSV-tag (YTDIEMNRLGK)
Xpress-tag (DLYDDDDK).

13. The method according to any one of claims 1 to 12, wherein the solubility tags are selected from

| | |
|---|---|
| Glutathione S-Transferase | GST |
| Small Ubiquitin-like Modifier | SUMO |
| Maltose Binding Protein | MBP |
| Fasciola hepatica 8 kDa antigen | FH8 |
| Thioredoxin | TRX |
| Solubility Enhancing Ubiquitous Tag | SNUT |
| Seventeen kilodalton protein | SKP |
| Monomeric bacteriophage T7 orc protein | MOCR |
| E coli secreted protein A | ESPA |
| N-utilization substance | NusA |
| IgG domain BO of Protein G | GB0 |
| IgG repeat domain ZZ of Protein A | ZZ |
| Mutated dehalogenase | HaloTag |
| Phage T7 protein kinase | T7PK |
| E. coli trypsin inhibitor | Ecotin |
| Calcium-binding protein | CaBP |
| Stress-response arsenate reductase | ArsC |
| N-terminal fragment of translation initiation | IF2-domain 1 |
| factor IF2 | |
| Stress-response protein | RpoA |
| Stress-response protein | SlyD |
| Stress-response protein | Tsf |
| Stress-response protein | RpoS |
| Stress-response protein | PotD |
| Stress-response protein | Crr |
| E. coli acidic protein | msyB |
| E. coli acidic protein | yjgD |
| E. coli acidic protein | rpoD |
| T7 phage tail | P17 |
| metal-binding protein | CUSF |
| 53-amino-acid-long N-terminal extension | NEXT |
| sequence | |

14. The method according to any one of claims 1 to 13, wherein each nucleic acid expression construct suitable for cell-free protein expression encodes a tripartite fusion protein, said nucleic acid molecule comprising:
a first nucleic acid moiety encoding one or more amphipathic protein(s) selected from the group consisting of Apolipoprotein A (Apo-A1, Apo-A2, Apo-A4, and Apo-A5), apolipoprotein B (ApoB), apolipoprotein C (ApoC), apolipoprotein D (ApoD), apolipoprotein E (ApoE), apolipoprotein F (ApoF), apolipoprotein L (ApoL), apolipoprotein M (ApoM), apolipoprotein M (ApoM) and a peptide self-assembly mimic (PSAM);
a second nucleic acid moiety encoding an integral membrane or hydrophobic protein; and
a third nucleic acid moiety encoding one or more solubility tag(s) in the form of water soluble expression decoy protein(s).

15. The method according to claim 14, wherein the left flank primers include a variety of solubility tags for screening the expression and solubility of the integral membrane or hydrophobic protein.

16. The method according to any one of claims 1 to 15, wherein the left flank and/or right flank primer further comprise protective elements that inhibit digestion of the left flank and/or right flank primers and the resulting expression construct by nucleases.

17. The method according to any one of claims 1 to 16, wherein the amplification of constructs uses modified nucleotides that can render the amplicon resistant to nuclease digestion or wherein the protective elements enable circularisation of the expression construct to thereby protect the expression construct from terminal nucleases.

18. The method according to any one of claims 1 to 17, wherein the amplification using the left and right flank primers uses 25-28 PCR cycles.

19. The method according to any one of claims 1 to 18, wherein the left flank primers are independently between 500 and 3000 nucleotides in length.

20. The method according to any one of claims 1 to 19, wherein the left flank primers are at least 1000 nucleotides in length.

21. The method according to any one of claims 1-20, wherein the forward adapter priming sequence and/or the reverse adapter priming sequence contain one or more restriction sites or homology arms to enable insertion into a cloning vector.

22. An expression construct or population of expression constructs prepared according to any one of claims 1-21.

23. A method of expressing a protein using a construct or population of constructs according to claim 22 using a cell-free system.

24. The method of claim 23 wherein the protein expression is performed on a digital microfluidic device containing an array of electrodes.

25. A kit comprising an expression construct or population of expression constructs according to claim 22 and components for cell-free protein expression.

26. A kit comprising a population of left flank primers and a single right flank primer for amplification of a nucleic acid wherein:
i. the left flank primers each comprise a promoter sequence, a sequence encoding for a ribosome binding site and one or more solubility tags, and at its 3′ end a sequence complementary to a nucleic acid to be amplified, wherein the population contains different solubility tags; and
ii. the right flank primer comprises a sequence coding for a detection tag, a sequence coding for a purification tag, a sequence encoding for a stop codon and, at its 3′ end, a sequence complementary to a nucleic acid to be amplified.

27. The kit according to claim 26 wherein the left flank primer ends with the A0 complementary sequence 5′-CTCGAGGTTCTGTTCCAAGGACCT-3′.

28. The kit according to claim 26 or claim 27 wherein the right flank primer ends with the B0 complementary sequence 5′-GAGAACCTGTACTTCCAGAGC-3′.

29. The kit according to claim 26 containing at least 8 left flank primers, wherein a first left flank has no solubility tag and the remaining 7 flank primers have the solubility tags: P17, CUSF, FH8, TRX, ZZ, SUMO, SNUT.
